(19) United States
(12) Patent Application Publication
(10) Pub. No.: US 2026/0242447 A1
(43) Pub. Date: Aug. 20, 2026

(54) ANTIVIRUS PROTEINS HAVING A KRINGLE 5 SUBUNIT

(71) Applicant: Creative BioTherapeutics, LLC, Gurnee, IL (US)

(72) Inventor: Donald J. Davidson, Gurnee, IL (US)

(21) Appl. No.: 19/084,149

(22) Filed: Mar. 19, 2025

Related U.S. Application Data
(63) Continuation of application No. 18/799,496, filed on Aug. 9, 2024, now abandoned.
Application No. 18/799,496 is a continuation of application No. 17/235,652, filed on Apr. 20, 2021, now Pat. No. 12,060,410.
(60) Provisional application No. 63/012,900, filed on Apr. 20, 2020.

(51) Int. Cl. C07K 14/81; A61K 38/00; C07K 14/705

(52) U.S. Cl. CPC C07K 14/8132; C07K 14/705; A61K 38/00

(57) ABSTRACT

COVID-19 results from the infection of the SARS-CoV-2 virus and has spread quickly to literally infected the world. Although coronavirus spike proteins can recognize a broad range of host cell-surface proteins, inhibiting spike protein binding to a survival factor called GRP78 results in a significant reduction in SARS-CoV-2 attachment, entry and replication in lung and kidney cells. This inhibition is accomplished with a novel type of inhibitor that potently blocks the binding of SARS-CoV-2 spike protein and whole virus to surface-bound GRP78. These novel GRP78 inhibitors also down regulate cytokines (IL10, IL6), immune co-inhibitory checkpoint proteins (PD-L1, B7H3, B7H4), and up regulate immune co-stimulatory proteins (MHC-II, CD-86) resulting in the reduction of the immune suppressive nature of infected lung alveolar epithelial cells in vitro and in vivo. Finally, these novel GRP78 inhibitors inhibit the hyperfibrinolysis of infected lung cells by reducing the activation of plasmin on cell surfaces.

### CROSS-REFERENCE TO RELATED APPLICATIONS

This application is a continuation of U.S. patent application Ser. No. 18/799,496, filed Aug. 9, 2024, which is a continuation of U.S. patent application Ser. No. 17/235,652, filed Apr. 20, 2021, issued as U.S. Pat. No. 12,060,410, which claims priority to U.S. Provisional Patent Application No. 63/012,900, filed Apr. 20, 2020. To the maximum extent permitted, as stated in U.S. Provisional Patent Application No. 63/012,900, priority is claimed based on the family of then co-pending patent applications, U.S. Provisional Patent Application No. 62/584,564, filed Nov. 10, 2017, and U.S. patent application Ser. No. 16/184,247, filed Nov. 8, 2018, published as US 2019-0142913 on May 16, 2019. The aforesaid publication and U.S. Pat. No. 10,905,750 are incorporated by reference as if fully set forth herein.

### INCORPORATION OF SEQUENCE LISTING

The contents of the xml file named “40133_029US—40133_22US—CREAT-002-02US-ST26.xml,” which was created on Mar. 19, 2025, and is 303,533 bytes in size, are hereby incorporated by reference in their entirety.

### DESCRIPTION OF RELATED ART

COVID-19 is an abbreviation from “coronavirus disease 2019” that is a result of an infection from a coronavirus called SARS-CoV-2. This viral infection has been declared a pandemic by the WHO and is considered by the CDC as a serious public health problem. Cases of COVID-19 are exploding worldwide and as of yet there is no approved cure. COVID-19 can result in severe respiratory illness in patients with pre-existing conditions and in older adults leading to permanent lung damage and death.

Currently, the use of two anti-malaria drugs, chloroquine and hydroxychloroquine, have both shown promise in preventing SARS-CoV-2 virus from infecting cells in the laboratory. Recently, in a small number of preliminary clinical trials against COVID-19 using hydroxychloroquine and an antibiotic showed that in the blood of treated patients the amount of virus was reduced much faster than in nontreated patients. These results are encouraging and even though the side-effects of heart and nerve damage as well as suicidal thoughts are manageable, they are still disturbing. These studies have also not yet shown that the patients lived longer or were more likely to recover with hydroxychloroquine. Finally, in a very recent report another treatment, Leronlimab, which is an antibody against CCR5 has shown positive results in 8 patients. CCR5 inhibitors have been shown to reduce the COVID-19 stimulated inflammatory cytokine storm in the lungs allowing for more time for recovery. Leronlimab is not a cure or a vaccine. Again, there is an urgent and unmet need for safe, effective new drugs to treat COVID-19.

The off-label therapies described above are being used in the clinic for COVID-19 patients but were not designed for specific inhibition of SARS-CoV-2 virus attachment, entry and replication. Despite this, these off label therapies have shown some desirable responses. Although some results look promising, many patients do not respond to these therapies and many deaths worldwide are still happening. New drugs that specifically target the SARS-CoV-2 virus infection are badly needed. Our work to create novel GRP78 inhibitors for anti-cancer and anti-immune suppression, could be one of the new targets and therapies for COVID-19 that is badly needed.

Understanding the mechanism of how viruses and host cell-surface proteins interact could help define their tropism, pathogenicity and lead to potential new targets for inhibition. For example, recent publications have described a role for surface-bound GRP78 during virus entry and replication. GRP78 was identified as a co-receptor for Coxsackievirus A9 and Dengue virus for attachment and entry. In addition, for the Japanese encephalitis virus (JEV), cell surface GRP78 is important for viral entry and critical for virus replication. Surface-bound GRP78 is also known to serve as an attachment factor for four betacoronaviruses, MERS-CoV, bCoV-HKU9, SARS-CoV, and SARS-CoV-2. FIG. 1 illustrates how coronaviruses use GRP78 as a co-receptor for attachment, internalization and replication. Virus infection of lung airway cells also results in the up regulation of GRP78 surface expression, which leads to further attachment and enhanced viral entry of infected cells.

Although coronavirus spike proteins can recognize a broad range of host cell-surface proteins, inhibiting GRP78, by either knockdown with siRNA, or cleavage with subtoxin A, or with an antibody, results in significant reduction in virus attachment, entry and replication. High expression of GRP78 was shown to be on the surface of stressed epithelial and endothelial cells along the human airways. Recently, it has been shown that cigarette smoke increased surface expression of GRP78 on stressed bronchial epithelial cells. Since COVID-19 has been shown to be worse in people that smoke, vape, have respiratory disease or are older, we suspect the expression of surface GRP78 on lung epithelial cells is significantly higher in this population. Even though expression of GRP78 alone was not enough to render nonpermissive cells susceptible to MERS-CoV infection, it has been shown that GRP78 is critical for viral entry and replication.

### BRIEF SUMMARY OF THE INVENTION

I have discovered that surface-bound GRP78 on A549 adenocarcinoma human alveolar basal epithelial cells and VERO epithelial cells up regulates immune co-inhibitory checkpoint proteins, PD-L1, B7H3, B7H4 and down regulates immune co-stimulatory proteins, MHC-II and CD86. I have also discovered that surface-bound GRP78 up regulates cytokines IL-10, IL6, on A549 adenocarcinoma human alveolar epithelial cells which results in the blunting of the immune response. I have created a class of novel and potent inhibitors that specifically bind to the N-terminal domain of GRP78 that block the binding of SARS-CoV-2 virus to GRP78 and completely reverses cytokine expression and the immune suppressive phenotype on lung epithelial A549 cells and VERO epithelial cells (FIG. 1B). Stressed epithelial cells up regulate surface bound GRP78 that not only acts as a co-receptor for SARS-COV-2 virus to assist in attachment, entry and replication, but also blunts the immune response against SARS-COV-2 virus and infected cells. The GRP78 inhibitor of the invention can significantly reduce the attachment, entry and replication of SARS-COV-2 virus as well as reduce the immune suppressive nature of infected lung alveolar epithelial cells in vitro and in vivo.

### DESCRIPTION

Dr. Dvorak published that a tumor is like a wound that won't heal. A very general analogy is that the same kind of mechanism exists for SARS-CoV-2 infection where the infection in the lungs is like a wound that won't heal. Viral infection induces enormous stress upon the infected cells and increases expression of GRP78 as happens in the tumor microenvironment (TME). The TME induces a pro-inflammatory, immune suppressive tumor cells similar to what is observed with viral infections on their target cells. In the co-pending GRP78 Antagonist application GRP78 inhibitors, Kr1Fc, K5Fc and K5 can block GRP78's interaction with cell surface receptors and decrease the immune suppressive, inflammatory nature of tumor cells. We now show for the first time that our GRP78 inhibitors, Kr1Fc, K5Fc and K5 can block the binding of SARS-CoV-2 spike protein to GRP78 with nM potency. Furthermore, our GRP78 inhibitors, Kr1Fc and K5, potently block whole live virus, pseudotyped SARS-CoV-2, attachment and entry into VERO kidney epithelial cells. Specifically, the co-pending GRP78 Antagonist application teaches:

A surface-bound GRP78 inhibitor blocks SARS-COV-2 spike protein attachment and entry. The co-pending GRP78 Antagonist application teaches inhibitors to GRP78 on endothelial and cancer cells. These inhibitors have now been tested against SARS-COV-2 virus binding to GRP78 and to human lung cells. A lead inhibitor, containing the kringle domain of ROR1 fused to a human IgG1 Fc domain (Kr1Fc), binds with high affinity to the N-terminal domain of GRP78. The invention teaches that Kr1Fc, K5Fc and K5 potently block the binding of SARS-COV-2 spike protein to GRP78. The disclosed GRP78 inhibitors are effective at blocking the attachment, entry and replication of SARS-COV-2 pseudotyped virus.

A new mechanism that promotes immune tolerance that is readily targetable. Over expression of GRP78 in stressed cells leads to a large increase in surface-bound GRP78. The invention teaches that surface-bound GRP78 on human A549 adenocarcinoma alveolar basal epithelial cells induces the expression of A) cytokines IL-10, and IL-6, B) immune co-inhibitory checkpoint proteins, PD-L1, B7H3, B7H4, and C) suppresses the expression of immune co-stimulatory proteins, MHC-II, and CD86. By blocking GRP78 binding to surface proteins with Kr1Fc, the immune-suppressive phenotype of A549 cells can be reversed. In the invention, inhibition of GRP78 binding to SARS-COV-2 SPIKE PROTEIN will lead to a decrease in viral load, cytokine storm and immune suppression associated with SARS-COV-2 infection.

Novel GRP78 inhibitors that bind to the N-terminal domain of GRP78 reduce surface GRP78 expression which is only expressed on stressed cells and not normal cells, leads to a safer therapy than other currently approved anti-viral therapies. The invention teaches potent inhibitors that bind tightly to the N-terminal domain of GRP78 resulting in the inhibition of SARS-CoV-2 virus binding. In the invention the inhibitors to surface-bound GRP78 are safe in CEREP receptor binding and in normal fibroblast proliferation assays. Previously practiced therapies being used against COVID-19 like hydroxychloroquine, bind weakly to SARS-CoV-2's receptor ACE2 on lung epithelial cells. The fact that ACE2 is expressed on several other normal cells and that hydroxychloroquine has such a weak binding affinity, supports the off-target side-effects of this drug seen in clinical trials. Another drug approved for treatment of SARS-CoV-2 virus is Remdesivir. Remdesivir is an adenosine analogue that blocks mitochondrial RNA polymerase essential for virus replication. However, there are several off-target toxicities in the gut and lungs with Remdesivir and it too must be used cautiously. This invention teaches GRP78, which is not expressed on the surface of normal cells is a safer and more effective target for therapies against COVID-19.

The invention also teaches that N-terminal GRP78 inhibitors block SARS-CoV-2 virus induced hyperfibrinolysis and coagulopat+hy (“clot storm”) through inhibition of plasmin generation (FIG. 2). Patients with severe COVID-19 have comorbidities like hypertension, heart disease, diabetes, and cancer that are known to have serious coagulopathies. Hyperfibrinolysis, reflected by elevated serum D-dimer levels, was present in 97% of the severe COVID-19 patients before death. Plasmin, a key player in hyperfibrinolysis and as such D-dimer levels, also enhances the virulence and pathogenicity of the SARS-CoV-2 virus by clipping a furin site on its envelope protein. Kr1Fc, K5Fc, and K5 can also block the activation of plasmin on lung epithelial and endothelial cell surfaces leading to reduced fibrinolysis, D-dimer formation and furin site cleavage. Not only will our GRP78 inhibitors significantly block SARS-CoV-2 virus attachment, entry and replication but they also reduce the cytokine storm, inhibit hyperfibrinolysis and increase the immune surveillance against SARS-CoV-2 virus (FIG. 2). The invention teaches N-terminal binding GRP78 inhibitors either alone or in combination with other therapies will significantly reduce the pathogenicity of COVID-19.

### BRIEF DESCRIPTION OF THE DRAWINGS

FIG. 1A is a schematic of SARS-CoV-2 virus attachment, entry and replication with and without GRP78 inhibitors.

FIG. 1B is a schematic of SARS-Cov-2 virus attachment, entry and replication with GRP78 inhibitors.

FIG. 2 is a schematic of SARS-CoV-2 virus inhibition by our novel class of GRP78 inhibitors.

FIG. 3A is an analysis of Kr1Fc (SEQ ID NO: 20) proteins purity, structure and binding to GRP78.

FIG. 3B is an analysis of K5Fc (SEQ ID NO: 2) proteins purity, structure and binding to GRP78.

FIG. 3C is an analysis of Kr1 (SEQ ID NO: 19) proteins purity, structure and binding to GRP78.

FIG. 3D is an analysis of K5 (SEQ ID NO: 1) proteins purity, structure and binding to GRP78.

FIG. 3E is an analysis of K5 (SEQ ID NO: 1) or KR1 (SEQ ID NO: 19) proteins purity, structure and binding to GRP78.

FIG. 3F is an analysis of proteins inhibitor binding on Human GRP78.

FIG. 4A is a schematic that shows that N-terminal GRP78 inhibitors block binding of GRP78 to SARS-CoV-2 spike protein.

FIG. 4B is a graph that show that N-terminal GRP78 inhibitors block binding of GRP78 to SARS-CoV-2 spike protein.

FIG. 5A is a series of graphs that show that GRP78 inhibitors block binding of PE-labeled SARS-CoV-2 spike protein to A549 lung cells.

FIG. 5B is two histograms that show that GRP78 inhibitors block binding of PE-labeled SARS-CoV-2 spike protein to A549 lung cells.

FIG. 6A is a series of graphs that show that GRP78 inhibitors prevent SARS-CoV-2 spike protein-PE binding to VERO cells.

FIG. 6B is two histograms that show that GRP78 inhibitors prevent SARS-CoV-2 spike protein-PE binding to VERO cells.

FIG. 7A is a series of graphs that show that Kr1Fc (SEQ ID NO: 20) and K5 (SEQ ID NO: 1) prevent binding of SARS-CoV-2 spike protein either with preincubation with VERO cells or added at the same time as the spike protein.

FIG. 7B is two histograms that show that Kr1Fc (SEQ ID NO: 20) and K5 (SEQ ID NO: 1) prevent binding of SARS-CoV-2 spike protein either with preincubation with VERO cells or added at the same time as the spike protein.

FIG. 8A is a series of graphs that demonstrate that N-terminal GRP78 inhibitors reduce the expression of surface-bound GRP78 on VERO cells.

FIG. 8B is two histograms of graphs that demonstrate that N-terminal GRP78 inhibitors reduce the expression of surface-bound GRP78 on VERO cells.

FIG. 9A is a series of graphs that demonstrate that N-terminal GRP78 inhibitors reduce the expression of surface-bound GRP78 on A549 lung cells.

FIG. 9B is two histograms of graphs that demonstrate that N-terminal GRP78 inhibitors reduce the expression of surface-bound GRP78 on A549 lung cells.

FIG. 10A is a schematic that shows that surface-bound GRP78 expression on VERO cells, which is significantly reduced by Kr1Fc (SEQ ID NO: 20) and K5 (SEQ ID NO: 1), is essential for SARS-CoV-2 spike protein internalization.

FIG. 10B is a series of graphs that show that surface-bound GRP78 expression on VERO cells, which is significantly reduced by Kr1Fc (SEQ ID NO: 20) and K5 (SEQ ID NO: 1), is essential for SARS-CoV-2 spike protein internalization.

FIG. 10C is histograms that show that surface-bound GRP78 expression on VERO cells, which is significantly reduced by Kr1Fc (SEQ ID NO: 20) and K5 (SEQ ID NO: 1), is essential for SARS-CoV-2 spike protein internalization.

FIG. 11A is a graph that demonstrates that GRP78 inhibition with N-terminal GRP78 binding proteins, Kr1Fc (SEQ ID NO: 20) and K5 (SEQ ID NO: 1), potently and significantly inhibit whole SARS-CoV-2 pseudotyped virus attachment and internalization on VERO cells.

FIG. 11B is a graph that demonstrates that GRP78 inhibition with N-terminal GRP78 binding proteins, Kr1Fc (SEQ ID NO: 20) and K5 (SEQ ID NO: 1), potently and significantly inhibit whole SARS-CoV-2 pseudotyped virus attachment and internalization on VERO cells.

FIG. 12A is a series of graphs which illustrate that K5 significantly augments co-inhibitory (PD-L1, B7H4) checkpoint protein and co-stimulatory (CD86, MHC-II) protein expressions induced by soluble GRP78 on A549 lung cells.

FIG. 12B is a histogram which illustrates that K5 significantly augments co-inhibitory (PD-L1, B7H4) checkpoint protein and co-stimulatory (CD86, MHC-II) protein expressions induced by soluble GRP78 on A549 lung cells.

FIG. 13A is a series of graphs that show that Kr1Fc (SEQ ID NO: 20) significantly augments expression of co-inhibitory (PD-L1, B7H4) checkpoint protein and co-stimulatory (CD86, MHC-II) protein expressions induced by soluble GRP78 on A549 lung cells.

FIG. 13B is a histogram that shows that Kr1Fc (SEQ ID NO: 20) significantly augments expression of co-inhibitory (PD-L1, B7H4) checkpoint protein and co-stimulatory (CD86, MHC-II) protein expressions induced by soluble GRP78 on A549 lung cells.

FIG. 14A and FIG. 14B are histograms that demonstrate that Kr1Fc (SEQ ID NO: 20) and K5 (SEQ ID NO: 1) inhibit soluble GRP78 induced cytokine expression of IL10 and IL6 from A549 lung cells.

FIG. 15A is a schematic that shows that Kr1Fc (SEQ ID NO: 20) and K5 (SEQ ID NO: 1) inhibit the activation of human plasminogen on VERO cell surfaces.

FIG. 15B is a histogram that shows that Kr1Fc (SEQ ID NO: 20) inhibit the activation of human plasminogen on VERO cell surfaces.

FIG. 16 is a graph that shows the mice treated with Kr1Fc (SEQ ID NO: 20) (60 mg/kg) or K5 (SEQ ID NO: 1) (90 mg/kg) every other day, intraperitoneally displayed no weight loses or overt toxicity.

FIG. 17 is a table showing GRP78 recognition binding sequences from several viruses compared to known GRP78 binding sequences from K5 (SEQ ID NO: 190) and Kr1 (SEQ ID NO: 191).

### DETAILED DESCRIPTION

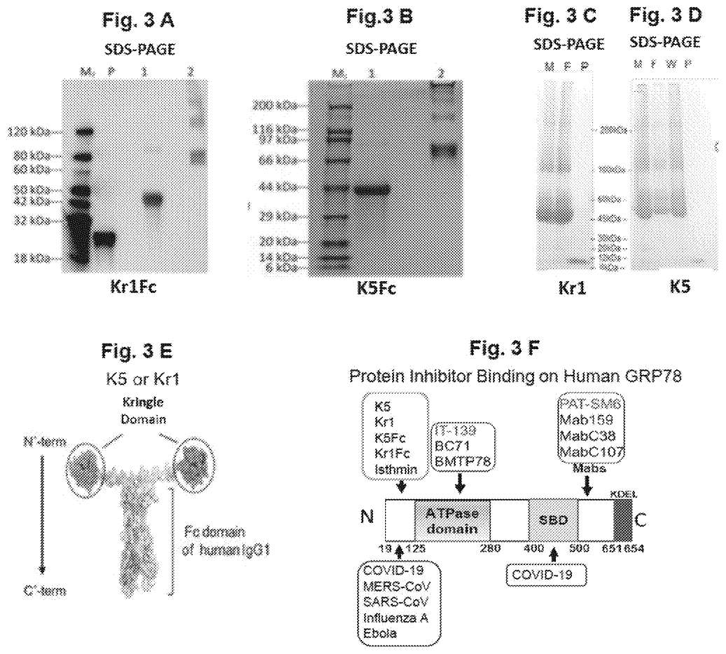

My previous publications, incorporated by reference, teach that the 5th kringle domain of human plasminogen (K5) (SEQ ID NO: 1) bound to surface GRP78 to induce inhibition of tumor angiogenesis and tumor growth. However, K5 (SEQ ID NO: 1) was not considered to be a good drug candidate due to its unknown mechanism of action and poor half-life in mice and monkeys (<20 min). By determining how soluble GRP78 binds to tumor cell surfaces, a novel GRP78 binding protein was identified and is called receptor tyrosine kinase-like receptor-1 (ROR1). Predictably, ROR1 has a kringle domain that is very similar (>70%) to K5. The ROR1 kringle domain, Kr1 (SEQ ID NO: 19), binds to GRP78 100Xs tighter (Kd=0.005 nM) than K5 (Kd=0.6 nM). The invention also shows that the ROR2 has a >70% homology to K5 and will have similar activity as ROR1 kringle domain. As shown in FIG. 3, the invention discloses novel kringle fusion protein that contains the ROR1 kringle domain, Kr1 (SEQ ID NO: 19), and a human IgG1 Fc domain. The addition of an IgG Fc domain to peptides has been shown to increase plasma half-life. We have now expressed the fusion proteins, Kr1Fc (SEQ ID NO: 20) and K5Fc (SEQ ID NO: 2) in Expi293F HEK cells and ExpiCHO cells and purified them over a Protein A column. The binding of the recombinant Kr1Fc (SEQ ID NO: 20), Kr1 (SEQ ID NO: 19), K5 (SEQ ID NO: 1), and K5Fc (SEQ ID NO: 2) proteins to benzamidine-agarose as a second purification column suggests that the kringle domains are folded properly. At the time of this writing, I have expressed and purified about 600 mgs of Kr1Fc (SEQ ID NO: 20), 2 mgs of K5Fc (SEQ ID NO: 2), 10 mgs of Kr1 (SEQ ID NO: 19), and 600 mgs of K5 (SEQ ID NO: 1). The references incorporated by reference discloses soluble GRP78 inhibitors Kr1Fc (SEQ ID NO: 20), K5Fc (SEQ ID NO: 2), Kr2Fc (SEQ ID NO: 34), and Kr1 (SEQ ID NO: 19) for use as anticancer agents.

### Binding Inhibition Example #1 GRP78 Inhibitors Kr1Fc, K5Fc and K5 inhibit SARS-CoV-2 spike protein binding to GRP78

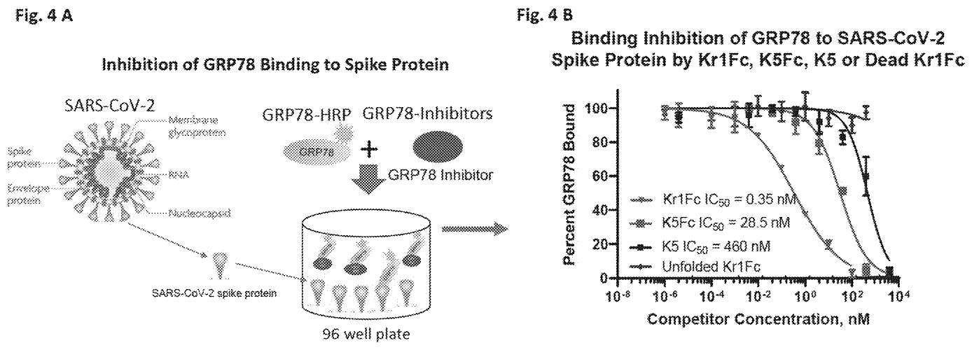

The prior art describes that GRP78 binds to the spike protein of SARS-CoV-2 virus. The prior art does not disclose what the affinity of binding was for SARS-CoV-2 spike protein with GRP78. Using an ELISA assay, this invention examines that GRP78-HRP binds to plate bound SARS-CoV-2 spike protein with a Kd of 293+35 nM. To determine if Kr1Fc, K5Fc and K5 can block this binding, this invention teaches to coat a 96 well plate with SARS-CoV-2 spike protein (100 nM) overnight at 4° C. The plate was then blocked with skimmed milk at room temperature for 30 min. HRP-labeled GRP78 (200 nM) is then added to the plates with various concentrations of Kr1Fc, K5Fc, K5 and a negative control, unfolded Kr1Fc. The plates were then incubated at room temperature for 2 hours. Finally, the wells were washed with PBS and then 1-step ultra TMB-ELISA reagent was added to each well and incubated at room temperature for 60 minutes and then read at 450 nM on a spectrophotometer. FIG. 4, shows that Kr1Fc, K5Fc and K5 inhibit GRP78 binding to the SARS-CoV-2 spike protein with IC50 values of 0.35 nM, 28.5 nM and 460 nM respectively. The unfolded Kr1Fc negative control showed no inhibition of SARS-CoV-2 spike protein binding to GRP78. This invention indicates that the invention GRP78 N-terminal binding inhibitors potently block GRP78 and SARS-CoV-2 spike protein binding.

### Binding Inhibition Example #2 GRP78 inhibitors Kr1Fc and K5 inhibit SARS-CoV-2 spike protein binding to A549 alveolar epithelial adenocarcinoma cells at 4° C.

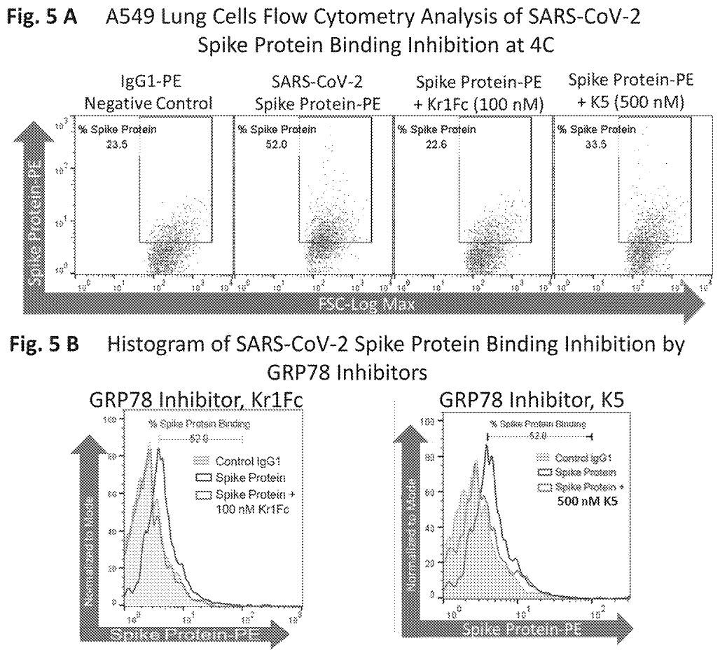

In FIG. 5, this invention examines if Kr1Fc and K5 could block binding of PE-labeled SARS-CoV-2 spike protein to A549 lung cells. A549 cells (50,000 cells/100 uL) were added to Eppendorf tubes in PBS. GRP78 inhibitors, at various concentrations, and 50 nM PE-labeled SARS-CoV-2 spike protein were added to the cells and incubated overnight at 4C to block internalization of the receptors. Cells were washed 2-times with PBS and run on a Guava PCA flow cytometer. The negative control used was a human IgG1-PE antibody. Flow cytometry analysis shows that SARS-CoV-2-PE spike protein bound to 52% of the A549 cells compared to control IgG1-PE antibody at 23% of cells. Kr1Fc at 100 nM significantly blocked the SARS-CoV-2-PE spike protein binding to A549 lung epithelial cells by greater than 99%, whereas K5 at 500 nM blocked the SARS-CoV-2-PE spike protein binding to A549 lung epithelial cells around 70%. These results indicate that GRP78 is important for SARS-CoV-2 virus binding to A549 lung epithelial cells.

### Binding Inhibition Examples #3 GRP78 inhibitors Kr1Fc and K5 inhibit SARS-CoV-2 spike protein binding to VERO cells at 37° C.

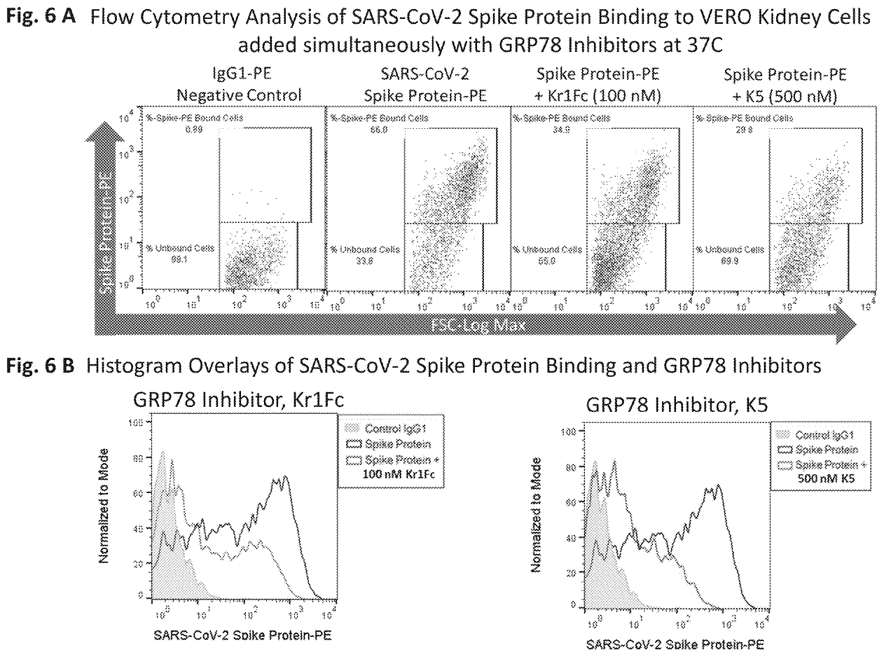

This invention examines if N-terminal GRP78 inhibitors could block the binding of SARS-CoV-2 spike protein on VERO monkey epithelial kidney cells. VERO cells are known to have high expression of the SARS-CoV-2 receptor, ACE2, and as such are very susceptible to SARS-CoV-2 virus infection. This invention teaches that both pre-incubation of VERO cells with GRP78 inhibitors and also by adding GRP78 inhibitors at the same time as the SARS-CoV-2 spike protein results in significant and potent inhibition of spike protein binding. VERO cells (50,000/100 uL) in PBS were added to Eppendorf tubes. Either Kr1Fc at 100 nM or K5 at 500 nM were added to half of the tubes of VERO cells for a pre-incubation time of 6 hrs. before 50 nM PE-labeled SARS-CoV-2 spike protein was added. In the other half of the tubes with cells, the PE-labeled SARS-CoV-2 spike protein was added at the same time as the GRP78 inhibitors listed above. The tubes of cells were incubated at 37C with mild shaking. After 24 hours, the cells were spun and washed twice with PBS. Fresh PBS was added to the cells and flow cytometry analysis of PE-labeled SARS-CoV-2 spike protein bound to VERO cells was detected on a Guava PCA flow cytometer. In this invention GRP78 inhibitors Kr1Fc and K5, displayed inhibition of SARS-CoV-2 spike protein binding with both a 6-hour pre-incubation or no pre-incubation with GRP78 inhibitors (FIGS. 6 & 7). Kr1Fc at 100 nM displayed greater than 90% inhibition of SARS-CoV-2 spike protein binding to VERO cells. K5 at 500 nM displayed an >80% inhibition of SARS-CoV-2 spike protein binding to VERO cells. The pre-incubation of GRP78 inhibitors on VERO cells leads to increased inhibition of SARS-CoV-2 binding.

### Internalization of Surface-Bound GRP78 Example #4 Surface-bound GRP78 is significantly decreased after Kr1Fc and K5 treatment

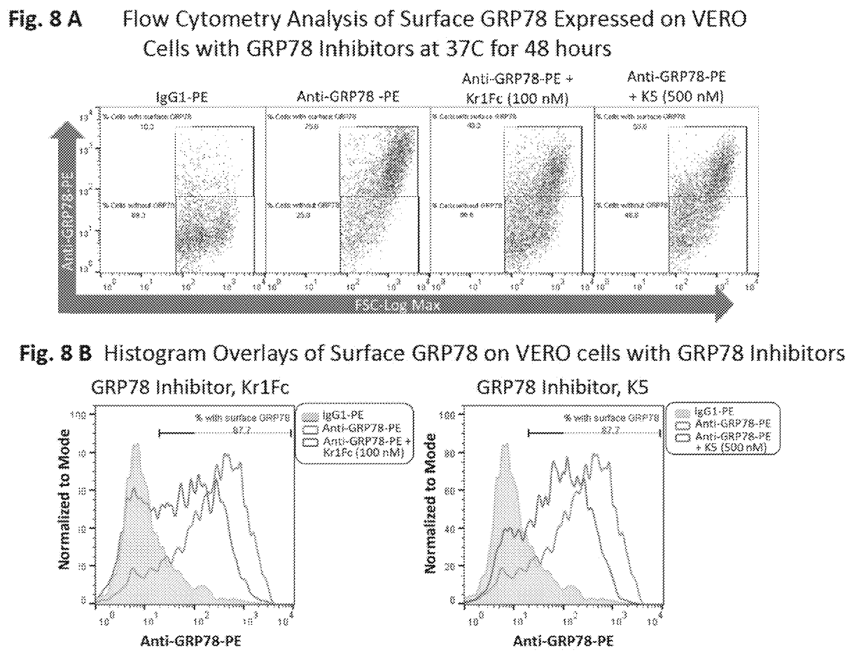

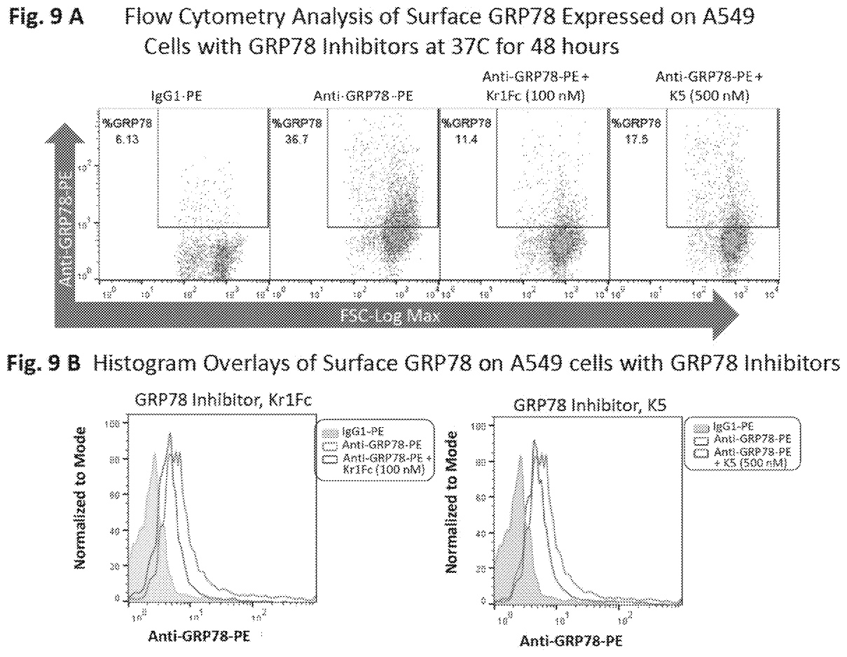

In this invention, Kr1Fc (100 nM), and K5 (500 nM) were added separately to Eppendorf tubes containing either 50,000 A549 cells or 50,000 VERO cells in triplicate. The cells and GRP78 inhibitors were incubated at 37C for 24 hours. The cells were pelleted by centrifugation and washed twice with PBS. Anti-GRP78 monoclonal antibody labeled with PE (1 mg/ml) was then added at 1 ul per tube. A negative control of a human IgG1-PE antibody was also added to a tube of untreated cells. After 1 hour incubation of the anti-GRP78-PE antibody at room temperature, cells were washed twice with PBS and analyzed for surface-bound GRP78 by a Guava flow cytometer. FIG. 8 demonstrates that the treatment of VERO cells with GRP78 inhibitors, Kr1Fc and K5, significantly reduced the expression of GRP78 on the surface of the cells. FIG. 9 shows that treatment of A549 lung cells with GRP78 inhibitors, Kr1Fc and K5 significantly reduced the concentration of surface-bound GRP78 by greater than 80%. Comparing the expression of surface-bound GRP78 on A549 and VERO cells, it is clear the VERO cells have a much higher level of surface-bound GRP78. Since these cells are more receptive for viral infection, this invention and data show that it is not only the levels of surface ACE2 but also GRP78 that is important for infectivity. Because these GRP78 inhibitors reduce the surface-bound GRP78 levels, does this result in the reduction of virus binding only or is GRP78 also responsible for internalization of SARS-CoV-2 spike virus.

### Internalization Inhibition Example #5 Kr1Fc and K5 inhibit pHrodo-red labeled SARS-CoV-2 spike protein internalization in VERO cells

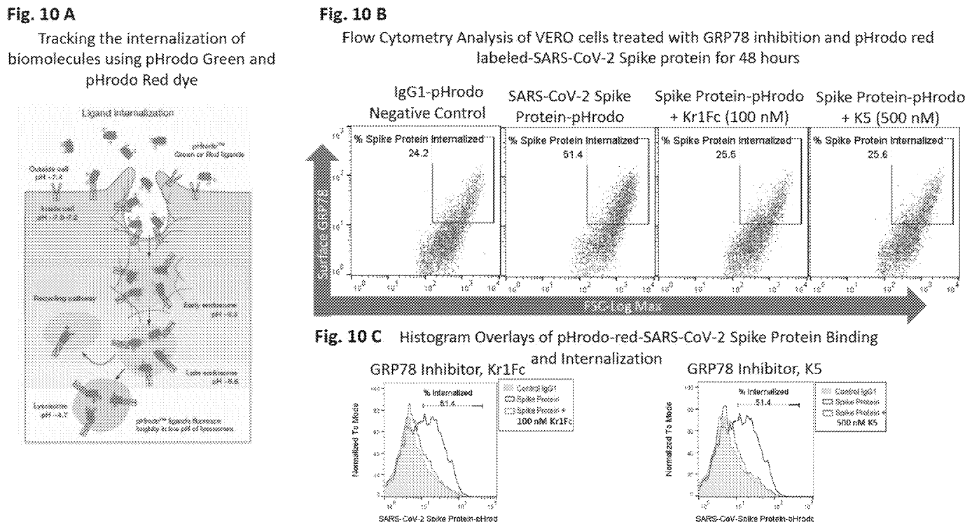

This invention examines if surface-bound GRP78 inhibition can inhibit binding of SARS-CoV-2 spike protein and block its internalization. As shown in FIG. 10A, SARS-CoV-2 spike protein was labeled with pHrodo-red dye, which has a weak fluorescence at pH 7 (cell surface), but has a strong fluorescence at pH 4 (inside cell) allowing for the detection of spike protein internalization. VERO cells were pre-incubated with GRP78 inhibitor, Kr1Fc and K5 for 6 hrs. After 6 hrs., Rodo-red labeled SARS-CoV-2 spike protein was added to the VERO cells and incubated at 37C with mild shaking for 24 hrs. Cells were then pelleted, washed twice and flow cytometry analysis was performed on a Guava PCA flow cytometer to detect inhibition of internalized spike protein. In FIG. 10B, this invention shows that Rodo-red labeled SARS-CoV-2 spike protein is internalized in about 52% of VERO cells in 48 hrs. at 37° C. When VERO cells are pre-incubated with the GRP78 inhibitor, Kr1Fc, or K5 the internalization of Rodo-red labeled SARS-CoV-2 spike protein is significantly inhibited by 90-95% similar to the Rodo-red labeled IgG1 antibody negative control. In both examples, A549 and VERO cells GRP78 inhibitors prevent SARS-CoV-2 spike protein internalization.

### Whole SARS-CoV-2 Virus Neutralization Example #6 GRP78 inhibitors, Kr1Fc and K5, neutralize SARS-CoV-2 pseudotyped virus infection of VERO cells

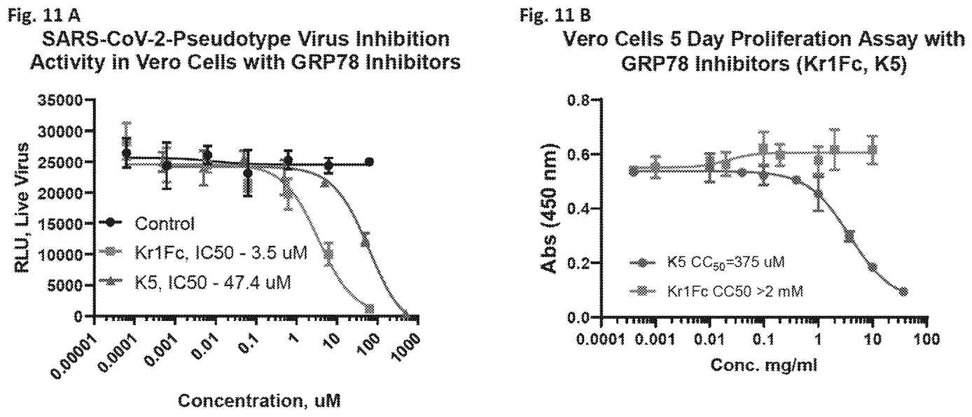

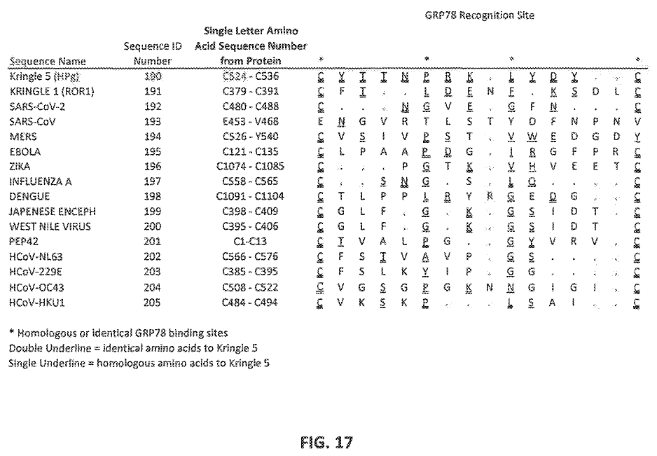

In this invention, modified SARS-CoV-2 virus assay was performed by IBT Bioservices (Rockville, MD). In this assay, a SARS-CoV-2 pseudotyped virus was generated by replacing the replication RNA piece from the SARS-CoV-2 virus with RNA encoding the Luciferase enzyme protein for detection. The remaining structural proteins (spike protein, envelope protein, and matrix protein and nucleocapsid protein) were left intact. This allows for SARS-CoV-2 (rVSV-SARS-CoV-2 (D614G)) pseudotyped virus to attach and internalize but not replicate. The invention teaches that Kr1Fc and K5 can prevent the full SARS-CoV-2 pseudotyped virus from attaching and entry into VERO cells. In FIG. 11A, two compounds demonstrate potent activity with IC50 values of 3.5 uM for Kr1Fc, and 47.4 uM for K5. The assay was performed by using eight dilutions from 0.5 nM to 500 uM for K5 and 0.01 nM to 62 uM for Kr1Fc. These dilutions were added to triplicate wells in a 96 well plate with VERO cells (1×10⁵ cells per well). Wells in accordance with the invention are infected with SARS-CoV-2 pseudotyped virus at 25,000-35,000 Relative Light Units in each well. Plates were incubated for 24 hours, and attached cells were washed and then each well was read for Luciferase activity using Bright-Glo Assay System Kit (Promega). The toxicity of the test compounds was also determined in parallel against VERO cells without virus (FIG. 10B). The 50% and 90% effective neutralization concentration (IC50, IC90) and 50% cell death concentration (cytotoxic, CC50) values are calculated by regression analysis to demonstrate efficacy. The selectivity index (SI90) (CC90 divided by IC90), which is indicative of the safety window between cytotoxicity and antiviral activity for 1 log inhibition was calculated and presented in FIG. 17. The higher the SI90 value, the more effective and safer the inhibitor.

### Immune Suppression Inhibition Example #7

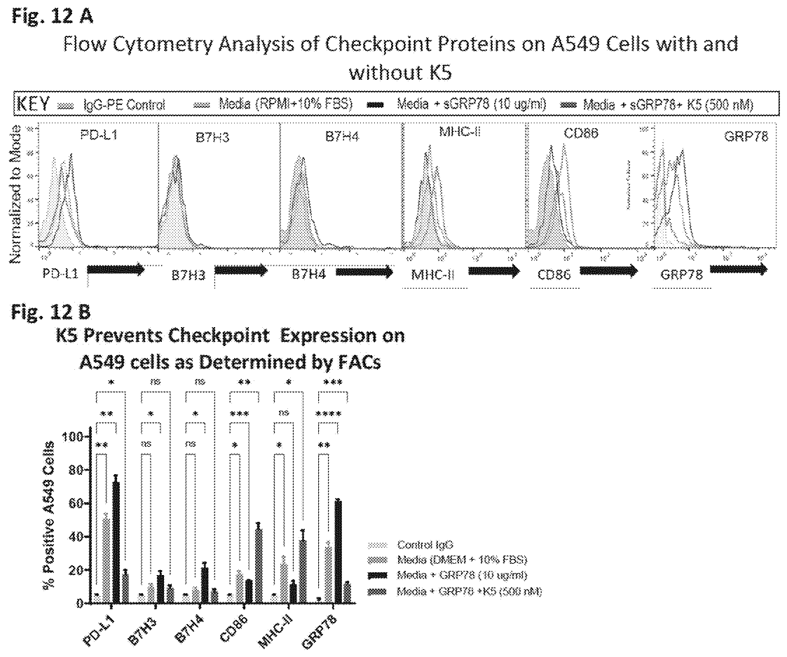

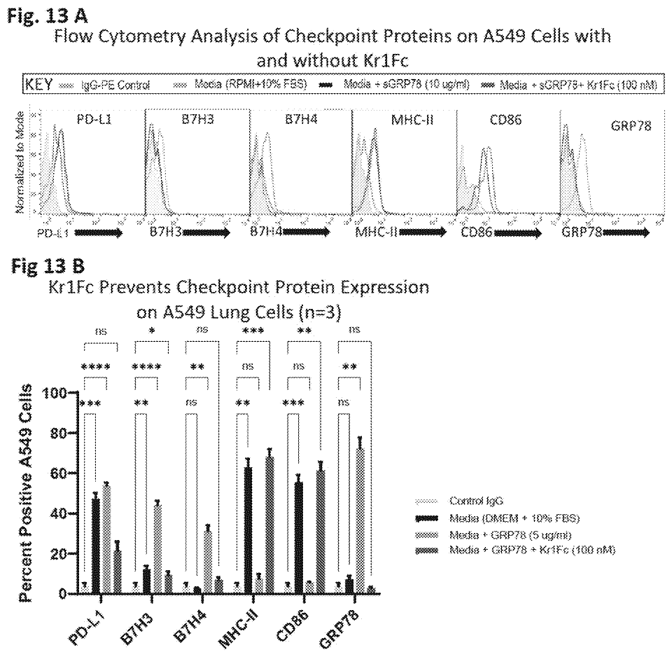

Kr1Fc, K5Fc and K5 reverses immune suppressive phenotype on adenocarcinoma alveolar lung epithelial cells (A549) induced by sGRP78 binding. This invention addresses whether soluble and surface bound GRP78, which has been shown to be stimulated during viral infections would augment checkpoint protein expression on lung cells similar to what has been reported with dendritic cells. To determine this, sGRP78 (5 ug/ml) was added to A549 cells and grown for 3 days i Kr1Fc at 37C/5% CO2. After 3 days, cells were fixed (not permeabilized), stained with fluorescently labeled antibodies and flow cytometry analysis was performed on co-inhibitor checkpoint proteins, PD-L1, B7H3, B7H4 and co-stimulatory proteins MHC-II, CD86. The present invention examines the expression of surface GRP78 with and without Kr1Fc, K5Fc and K5 inhibitors. We chose a concentration of 5 μg/ml sGRP78 because it has been shown that sGRP78 circulates in cancer and Rheumatoid Arthritis patients around this concentration. FIGS. 12 and 13 show that sGRP78 induced a significant increase in expression of immune inhibitory checkpoint proteins PD-L1, B7H3, B7H4 and decreased expression of immune stimulatory proteins MHC-II and CD86 on A549 cells. Kr1Fc and K5 completely reversed this immune-suppressive phenotype.

### Cytokine Expression Inhibition Example #8

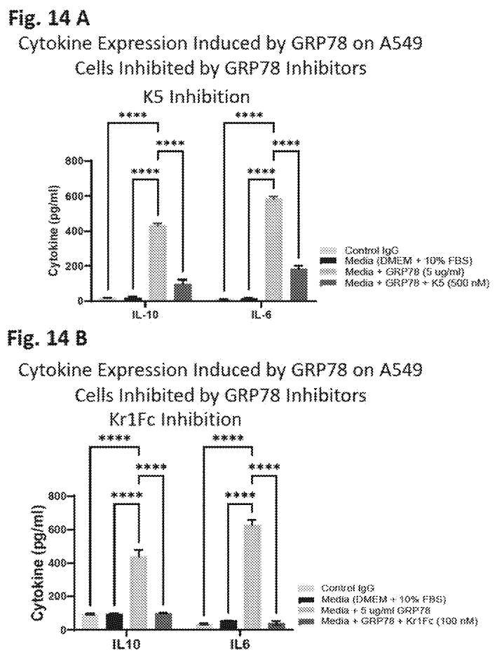

Previously, we have shown that soluble GRP78 induces cytokine expression on tumor cells. In many ways, viral infection mimics the tumor microenvironment, as such we determined that soluble GRP78 could up regulate expression of IL10 and IL6 on A549 lung cells. In FIG. 14, we now show that Kr1Fc and K5 inhibit soluble GRP78 induced cytokine expression of IL10 and IL6 from A549 lung cells. A549 cells (50,000) were added to Eppendorf tubes in full DMEM media. In half the tubes, soluble GRP78 (5 ug/ml) was added along with either K5 (500 nM) or Kr1Fc (100 nM). An IgG human antibody was used as a negative control. After incubation for 3 days at 37C with mild shaking, the cells were spun down and the supernatant was tested using either an IL10 or IL6 ELISA assay kits (R&D Systems). The protocol for each kit was followed per manufacturer instructions and pg/ml of each cytokine at each condition on A549 cells with and with GRP78 inhibitors was calculated from standard curves. A significant and dose dependent inhibition of cytokines expression from A549 cells was observed with Kr1Fc and K5 GRP78 inhibitors.

### Plasminogen Activation Inhibition Example #9 Kr1Fc and K5 block the hyperfibrinolysis induced by plasmin formation

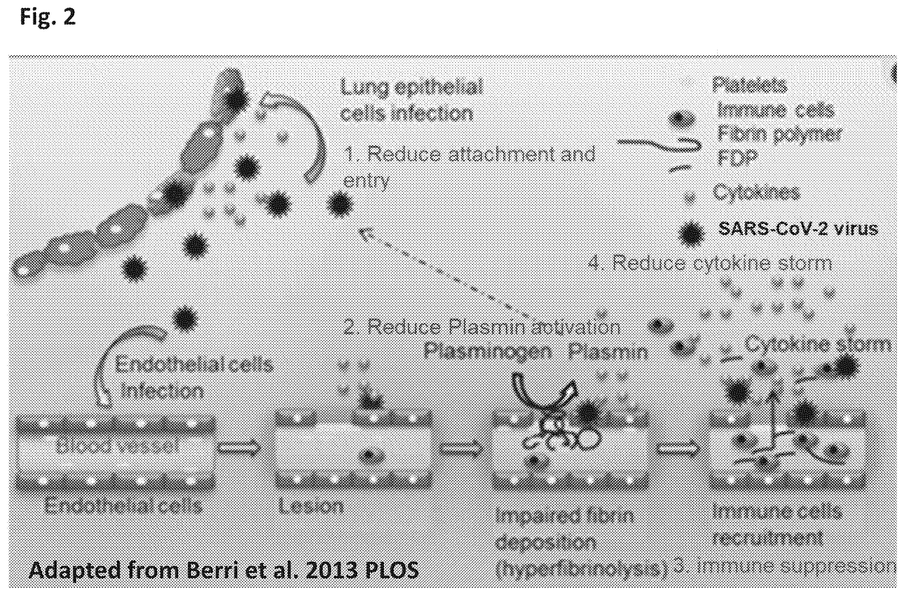

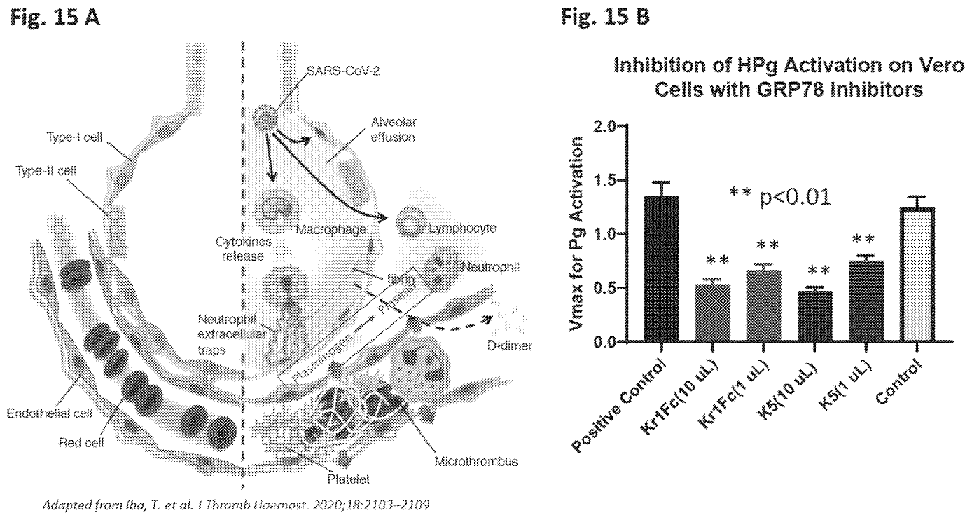

People with diabetes, hypertension, lung cancer and heart disease have a higher risk of being infected by SARS-CoV-2 virus and a greater chance of dying. The leading causes of death from COVID-19 is hemorrhage or bleeding disorders and that one of the characteristics of the disease is overactivity of the system responsible for removing blood clots (hyperfibrinolysis). This aberrant coagulopathy is caused by elevated levels of plasminogen leading to plasmin. Recent publications show that in all of the comorbidities that cause worse outcomes for people with COVID-19, elevated levels of plasmin have been found to be a common factor. Plasminogen normally circulates in blood as an inactive protein. Once an injury or a lesion occurs (FIG. 2, FIG. 15A (red)), plasminogen is activated to its active form called plasmin. Plasmin then dissolves blood clots at the lesions by clipping fibrinogen resulting in fibrinogen fragments called D-dimers. More than 97% of people hospitalized with severe COVID-19 have increased levels of D-dimer. D-dimer levels are associated with the amount of virus detected in the body and continue to rise as the severity of COVID-19 increases. In COVID-19 survivors, D-dimer levels decreased to control levels. Higher-than-normal levels of plasmin and D-dimers can lead to severe bleeding. Several publications have shown that plasminogen binds to surface-bound GRP78 through one of its kringle domains, kringle 5 (K5). In fact, GRP78 binding to plasminogen increases its rate of activation to plasmin significantly. Since our GRP78 inhibitors, Kr1Fc, K5Fc and K5, consists of the plasminogen K5 kringle domain or a similar kringle domain (Kr1), we can show that these inhibitors significantly decrease the activation of plasminogen to plasmin on lung cells by over 70% (FIG. 15). This decrease in plasmin formation should result in reduced hyperfibrinolysis and D-dimer formation reducing the bleeding disorders observed in severe COVID-19 patients.

### Safety Profile Example #9 Kr1Fc, and K5 show no adverse binding to receptors and ion channel proteins or toxicity on primary human cells

To identify possible toxicities, a receptor binding profile assay with 75 receptor and ion channels, and a cytotoxicity assay with 5 human primary cell lines (validated from single donor sources) with Kr1Fc (10 uM) or K5 (100 uM) were determined by CEREP/Eurofins. No specific binding or toxicity was observed, indicating that Kr1Fc exhibits a safe, selective biochemical profile and is unlikely to have adverse effects in vivo.

### Safety Profile Example #10 No weight loses or overt toxicity was observed in mice treated GRP78 inhibitors Kr1Fc and K5 at 60 mg/kg and 90 mg/kg respectively

This invention examines the toxicity of GRP78 inhibitors Kr1Fc and K5 in BALB/c mice 8 weeks old. Three groups of mice were dosed intraperitoneal (i.p.) in a volume of 10 mL/kg scaled to the body weight of each individual animal. Treatment groups were as follows: Group 1 received vehicle (PBS pH 7.2).
Group 2 received Kr1Fc at 60 mg/kg, i.p., every other day (qod) until day 26.
Group 3 received K5 at 90 mg/kg every other day (qod) until day 26.

Animals were weighed daily on Days 1-5, and then twice weekly until day 26. The mice were observed frequently for overt signs of any adverse, treatment-related (TR) side effects, and clinical signs were recorded when observed. Individual body weight was monitored as per protocol, and any animal with weight loss exceeding 30% for one measurement or exceeding 25% for three consecutive measurements was euthanized as a TR death. Group mean body weight loss was also monitored according to Charles River Discovery Services protocol. Acceptable toxicity was defined as a group mean body weight (BW) loss of less than 20% during the study and no more than 10% TR deaths. Deaths were classified as TR if it was attributable to treatment side effects as evidenced by clinical signs and/or necropsy.

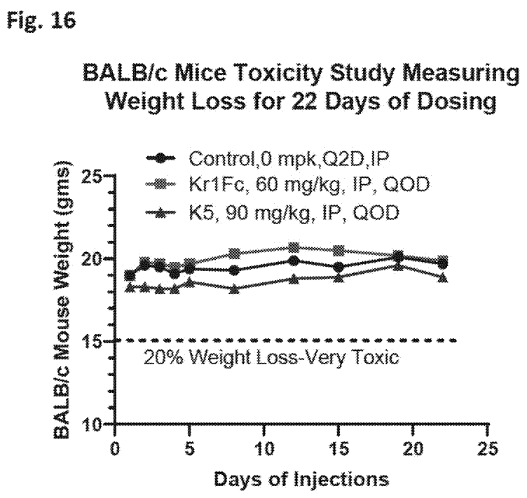

FIG. 16 shows that Kr1Fc and K5 showed no overt toxicity or weight loss in mice dosed as listed above. These results support the CEREP/Eurofins in vitro toxicity testing from above and suggest that the invention listed types of GRP78 inhibitors will be safe, with low toxicity in clinical studies.

### Chemistry, Manufacturing, and Controls (CMC) Aspects for the Development of K5Fc, K5 and Kr1Fc.

According to the invention the practitioner will express and purify Kr1Fc, K5Fc, K5 and perform CMC assays to validate the purity, potency, and efficacy of Kr1Fc, K5Fc and K5 lots for in vitro and in vivo SARS-CoV-2 virus inhibition studies. Currently, Kr1Fc, K5Fc and K5 are transiently expressed in Expi293 and ExpiCHO cells. Mammalian cell expression is necessary due to the need for a correctly folded kringle domains and glycosylation of the Fc domain. In accordance with the invention stable CHO clones expressing Kr1Fc, K5Fc and K5 will be created and banked, for regulatory filing. In accordance with the invention a practitioner will validate assays for purity, potency and identity of lots of Kr1Fc, K5Fc and K5. To validate Kr1Fc's, K5Fc's and K5's lot-to-lot quality, a negative control of denatured Kr1Fc and a positive standard lot of Kr1Fc are banked and used in each assay for comparisons.

### Statistical Analysis:

In accordance with the invention assays in Chemistry, Manufacturing, and Controls (CMC) aspects are run in triplicate on two separate occasions by two different investigators. The average mean value for absorbance, density and EC50 values plus the standard deviation are calculated for all assays. Any compound lot that is significantly (p<0.05, by student's two-tailed t-test assuming normal distribution and equal variance) different than our control standard lot will not be used for further testing.

### Lot to Lot Purity, Potency and Variability:

According to the invention the purity for Kr1Fc, K5Fc and K5 lots are determined by practicing 280 nm OD measurements for drug concentration, densitometry analysis (GelQuant) of Coomassie-stained SDSPAGE gels for drug purity and mass spectrometry analysis (fee for service) for drug quality. These assays are used to ensure the desired quality of Kr1Fc is maintained. For all in vitro studies, negative controls are Kr1Fc mixed with denatured, dead Kr1Fc at various ratios (10% to 90%) to define the limits of each assay and for a positive control, a master lot of Kr1Fc is set aside for comparison in all assays between different lots.

According to the invention the potency of Kr1Fc, KSFc and K5 lots are determined using a direct binding ELISA assay between Kr1Fc, or KSFc, or K5 and GRP78. Full-length his-tagged GRP78 (StressMart) at 100 nM in PBS is attached to nickel-coated 96 well plates (Pierce). Various concentrations of inhibitors between 0.5 to 50 nM are bound at 4° C. Plates are blocked with 10% BSA and washed with PBS containing 0.05% Tween®-20 detergent per manufacturer's instructions (Pierce). Finally, a mouse anti-human Fc HRP-labeled antibody is added, and detection of bound Kr1Fc, K5Fc or K5 is assessed with the TMB ELISA (Sigma) reagent. A secondary binding assay for Kr1Fc, K5Fc, and K5 lots are performed measuring Kr1Fc competition binding between SARS-COV-2 spike proteins and GRP78, as was described in our results (FIG. 4).

According to the invention functional efficacy of Kr1Fc lots is determined by examining immune co-inhibitory checkpoint (PD-L1, B7H3, B7H4) and immune co-stimulatory (CD86, MHC-II) protein expression. As described in FIG. 5, sGRP78 (5 ug/ml) are added to lung epithelial adenocarcinoma A549 cells and grown for 3 days+Kr1Fc at 37C/5% CO2. After 3 days, cells are removed by Versene, fixed, and then stained with fluorescently labeled antibodies and flow cytometry analysis are performed.

Assays that have a signal-to-noise ratio greater than two-fold and are able to detect >10% impurities, and assays with <200 nM EC(50) values for potency and functional assays are considered valid.

With respect to drug purity and identity, mass spectrometry can detect impurities below 5% reliably. The two-fold signal-to-noise requirement has been met for all assays except the functional checkpoint protein expression assays. An alternate approach is to test for protein phosphorylation in signaling pathways with A549 cells using Kr1Fc. Protein phosphorylation assays with signaling pathways through PI3K, SMAD2/3, STAT3 are known in the prior art. These signaling pathway assays lead to a rapid and accurate method to test for activity and functionality in lots of Kr1Fc.

According to the invention efficacy of GRP78 inhibitors, Kr1Fc, K5Fc, and K5, for blocking SARS-CoV-2 spike protein binding and internalization effects on lung epithelial A549 and VERO cells has been determined and validated.

The disclosed Kr1Fc, K5Fc and K5 inhibitors potently block binding of the SARS-CoV-2 spike protein to GPR78 (FIG. 4A). Much like tumors, viral infections create an inflammatory, immune evasive microenvironment. The references incorporated by reference shows that stressed cells relocate GRP78 to cell surfaces. Surface-bound GRP78 induces immune suppression by up regulating expression of co-inhibitory checkpoint proteins and down regulating expression of co-stimulatory proteins. GRP78 also increases the expression of inflammatory cytokines like IL-10, and IL-6. By blocking GRP78's activity and binding on lung, kidney epithelial and endothelial cell surfaces with our GRP78 inhibitors, one practicing the invention can completely reverse the immune suppressive, inflammatory nature of human alveolar lung epithelial adenocarcinoma A549 cells, VERO epithelial and endothelial cells. In addition, studies show that the blocking of the N-terminal domain of GRP78 reduces the attachment, entry and replication of several types of viruses including betacoronaviruses. As such, we show in these studies that our GRP78 inhibitors, which bind to the N-terminal domain of GRP78, will produce similar effects as siRNA or an N-terminal GRP78 antibody against the SARS-CoV-2 virus.

According to the invention neutralization activity against full pseudotyped SARS-CoV-2 virus (rVSV-SARS-CoV-2 (D614G)) of Kr1Fc and K5 are able to prevent the virus from attaching and entry into VERO cells grown in culture. Two compounds demonstrate potent activity with IC(50) values of 3.448 uM for Kr1Fc, and 47.35 for K5. Eight dilutions from 0.5 nM to 500 uM for K5 and 0.01 nM to 62 uM for Kr1Fc were added to triplicate wells with cells. Wells in accordance with the invention are infected with SARS-CoV2 pseudotyped virus at 25,000-35,000 Relative Light Units to each well. Plates were incubated for 24 hours, attached cells were washed and then each well was read for Luciferase activity using Bright-Glo Assay System Kit (Promega). The toxicity of the test compound was also determined in parallel against VERO cells without virus. The 50% and 90% effective neutralization concentration (IC50, IC90) and 50% cell death concentration (cytotoxic, CC50) values are calculated by regression analysis to demonstrate efficacy. The selectivity index (SI50) (CC50 divided by IC50), which is indicative of the safety window between cytotoxicity and antiviral activity was calculated and presented in Table 1. The higher the SI50 value, the more effective and safer the inhibitor.

In accordance with the invention potent inhibition of virus attachment is demonstrable. GRP78 is surface-bound in stressed and tumor cells, in accordance with the invention the practitioner may add GRP78 to the media of A549 and VERO cells to produce a cell line for virus attachment and internalization. In accordance with the invention K5, K5Fc and Kr1Fc will reduce surface-bound GRP78 by >90%. In accordance with the invention, the reversal of immune suppression, observed with A549 cells treated Kr1Fc, demonstrates a response which could reduce viral pathology in vivo.

In accordance with the invention Kr1Fc will inhibit an adapted SARS coronavirus in an acute respiratory distress syndrome lethal mouse model (BALB/c mice). SARS-COV-2 virus creates a severe acute respiratory syndrome disease that is highly lethal. To date there have been no drugs directly approved for curing betacoronavirus infections. Part of the reason for this is due to the lack of appropriate animal models for drug testing. Researchers are testing multiple animal models in macaques, marmosets, hamsters, cats, and ferrets with SARS viruses but few mimics the SARS-CoV-2 virus pathogenicity. Recently, Day et al. have adapted and characterized a new strain of SARS-CoV (v2163) that targets lungs and is highly lethal in BALB/c mice. This model largely mimics the human COVID-19 disease. Because of the low expense, ease of handling and minimal amount of drug required, this model when practiced in accordance with the invention demonstrates GRP78 inhibitor with a human SARS-CoV virus. Since mouse and human GRP78 are 98% identical, studies of this nature demonstrate Kr1Fc drug will block GRP78's activity in mice as well as humans resulting in a greatly reduced infection of lung cells.

### EXPERIMENT

In accordance with the invention the anti-viral activity of Kr1Fc and K5 is demonstrated using a mouse adapted strain of the Urbani SARS-CoV called V2163. Half male and half female BALB/c mice are inoculated with 50 uL containing 10⁴ CCID50 (Cell culture infectious dose 50% endpoint) of SARS-CoV-V2163 virus by intranasal (i.n.) delivery. Four groups of mice (10 mice per group) are given 100 mg/kg/day K5, 50 mg/kg/day Kr1Fc, and PBS, pH 7.4 negative control, and a positive control for this model used meeting the standards of the Institution for Antiviral Research. All Kr1Fc and K5 samples are dosed i.p., QD between shoulder blades 16 hours prior to i.n. infection for the next 7 days. Mice are observed daily, and group weights are taken throughout the study. Mice are observed for death up to day 21 post virus exposure. Animals that lose greater than 30% of their initial body weight are humanely euthanized and the day of euthanasia designated as the day of death. Lungs from sacrificed mice are observed for gross pathology and discoloration and assigned a score ranging from 0 (normal appearing lung) to 4 (maximal plum coloration in 100% of lung). Mouse lung samples from each test group are pooled and homogenized in MEM solution and assayed for A) infectious virus using the virus yield assay, B) cytokine analysis (IL-6, IL-1 and IL-10) and immune cell infiltrates (NK, Macrophage, T-cell and DCs). Virus titer, cytokine concentration and immune cell numbers are compared to controls by analysis of variance on log transformed values assuming equal variance and normal distribution. A significant (p<0.05) improvement in survival with Kr1Fc or K5 treatment compared to PBS negative control and a significant decrease in viral-induced CPE in the lungs of provides a foundation for further studies.

As described in the references incorporated by reference K5 has a half-life around 20 min and an MTD greater than 660 mg/kg in mice and monkeys. The more potent GRP78 inhibitors, Kr1Fc, and K5Fc in accordance with the invention and publication of proteins with an added Fc domain have improved half-lives and may be advantageous over the individual kringle domains. In addition, combination therapy with an ACE2 inhibitor and our GRP78 inhibitor could demonstrate synergistic effects.

In accordance with the invention, the conclusions described are demonstrated by statistical analysis. More specifically, graph generation and statistical analysis are done on GraphPad Prism 7.0 software. Statistical comparisons are performed two-way ANOVA with Bonferroni posttest by Student's two tailed t-test, assuming normal distribution and equal variance, where differences are considered significant at p<0.05. Power analysis with a Wilcoxon-Mann-Whitney test for a two-sided unpaired sample power analysis using the G*Power 3.1.9.4 program is used to determine the number of mice needed. The group size (n=10) is powered to detect decreases of at least 30% in the number of metastases between control and 10 mg/kg Kr1Fc treated-groups, assuming a coefficient of variation equal to 1.5 (as suggested by projected/anticipated data), and using a two-sample t-test for log normal data with 80% power and a significance level of 0.05. Maximum day of death (MDD), cytokine values, and gross lung scores are analyzed by Mann-Whitney pairwise comparisons or the Kruskal-Wallis test followed by Dunn's multiple comparison test as applicable. Raw survival numbers are compared by the Fisher exact test. Survivor curve analysis are done using the Kaplan-Meier method and a log rank test. When that analysis revealed significant differences among the treatment groups, pairwise comparisons of survivor curves are analyzed by the Gehan-Breslow-Wilcoxon test, and the relative significance adjusted to a Bonferroni-corrected significance threshold for the number of treatment comparisons made. Differences in percent weight loss are tested by one-way ANOVA with Newman-Keuls multiple comparison test, assuming equal variance and normal distribution. For lung titer data, we will perform a KS test for normality, then use non-parametric Kruskal-Wallis test with Dunn's multiple comparison test for groups that are not normally distributed, and a one-way ANOVA with Newman-Keuls multiple comparison test for groups that are normally distributed.

### DETAILED DESCRIPTION OF THE FIGURES

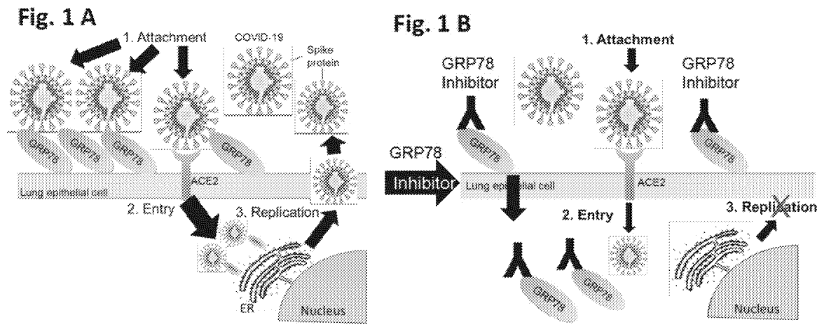

FIG. 1 are schematics of SARS-CoV-2 virus attachment, entry and replication with and without GRP78 inhibitors. FIG. 1A shows stress induces GRP78 to translocate to the cell surface. 1) SARS-CoV-2 virus attaches to GRP78 and ACE2. 2) GRP78 enhances SARS-CoV-2 virus entry into the lung epithelial cells. 3) GRP78 increases replication and secretion of SARS-CoV-2. FIG. 1B shows GRP78 inhibitors blocks a significant amount of SARS-CoV-2 virus binding and reduces surface-expression of GRP78, which then leads to a reduced amount of SARS-CoV-2 virus entry into the cells. Finally, the lack of GRP78 on cell surfaces inhibits SARS-CoV-2 virus attachment, internalization and replication. The X indicates that blocking GRP78 binding on the cell surface blocks viral replication

FIG. 2 is a schematic of SARS-CoV-2 virus inhibition by our class of GRP78 inhibitors. This invention shows that N-terminal GRP78 inhibitors block 1) SARS-CoV-2 virus attachment and entry, 2) reduces hyperfibrinolysis by blocking the conversion of plasminogen to plasmin on lung cell surfaces, 3) reverses immune suppression by reducing checkpoint protein expression on lung and endothelial cells and 4) reduces the virus induced cytokine storm by reducing IL10 and IL6 cytokine expression.

FIG. 3 is an analysis of Kr1Fc, K5Fc, Kr1 and K5 Proteins Purity, Structure and Binding. FIGS. 3A-D are image of SDS-PAGE analysis showing A) Kr1Fc, B) K5Fc, C) Kr1 and D) K5 proteins expressed in 293F [EK cells. Kringle proteins were purified by either protein A-agarose or benzamidine-agarose. Lane markers are Mw: Protein marker, Lane 1: Reducing conditions, Lane 2: Nonreducing conditions, M: expression media, F: flow through, W: wash, P: purified protein. A FIG. 3E is a graphic depiction of Kr1-Fc and K5Fc fusion proteins with 2 kringle domains fused to a human IgG1 Fc domain. A FIG. 3F is a schematic illustration of human GRP78 regions where published GRP78 protein inhibitors and the invention proteins interact. Known and predicted binding sites of viruses to GRP78 protein are shown underneath the GRP78 schematic. (ATPase domain SBD: substrate binding domain, KDEL; 4 residue C-terminal peptide).

FIG. 4 shows that N-terminal GRP78 inhibitors block binding of GRP78 to SARS-CoV-2 spike protein. FIG. 4A is a Schematic of a binding assay with full-length SARS-CoV-2 spike protein, HRP-labeled GRP78 and GRP78 inhibitors in a 96 well plate. FIG. 4B is a graph showing data that N-terminal GRP78 inhibitors, Kr1Fc, K5Fc, and K5 can potently block GRP78 binding to SARS-CoV-2 spike protein. Full length SARS-CoV-2 spike protein was bound to a 96 well plate overnight at 4° C. After blocking and washing, HPR-labeled GRP78 and various concentrations of Kr1Fc, K5Fc, K5 and a negative control, unfolded Kr1Fc were added to plates and incubated for 2 hrs. Wells were washed and TMB reagent was added and absorbance measured by manufactures protocol. Each point is an average of triplicate wells.

FIG. 5 shows that GRP78 inhibitors block binding of PE-labeled SARS-CoV-2 spike protein to A549 lung cells. SARS-CoV-2 spike protein-PE (50 nM) was added to 50,000 A549 lung cells with and without Kr1Fc (100 nM) or K5 (500 nM). Cells were incubated overnight at 4° C. to block internalization of the SARS-CoV-2 spike protein-PE. Cells were washed 2X with PBS and run on a Guava PCA flow cytometer. Control was a human IgG1-PE Ab. FIG. 5A shows Raw flow cytometry plots and FIG. 5B is a histogram overlay analysis of A549 cells with bound SARS-CoV-2 spike protein-PE competed with GRP78 inhibitors demonstrate that Kr1Fc and K5 block SARS-CoV-2 spike protein-PE binding to A549 lung cells by over 99% and 65% respectively.

FIG. 6 shows that GRP78 inhibitors prevent SARS-CoV-2 spike protein-PE binding to VERO cells. VERO cells were used due to their high expression of ACE2 and their ability to be very susceptible to SARS-CoV-2 virus infectivity. SARS-CoV-2 spike protein-PE (50 nM) was added to 50,000 VERO kidney cells with and without Kr1Fc (100 nM) or K5 (500 nM). Cells were incubated overnight at 37° C. Cells were then washed 2X with PBS and run on a Guava PCA flow cytometer. Control was a human IgG1-PE Ab. FIG. 6 A is dot blot flow cytometry analysis and FIG. 6B is a histogram overlay of VERO cells that bound SARS-CoV-2 spike protein-PE with and without Kr1Fc and K5. From the data it is clear that Kr1Fc (100 nM) and K5 (500 nM) significantly reduced the binding of SARS-CoV-2 spike protein-PE to the surface of VERO cells by about 50%.

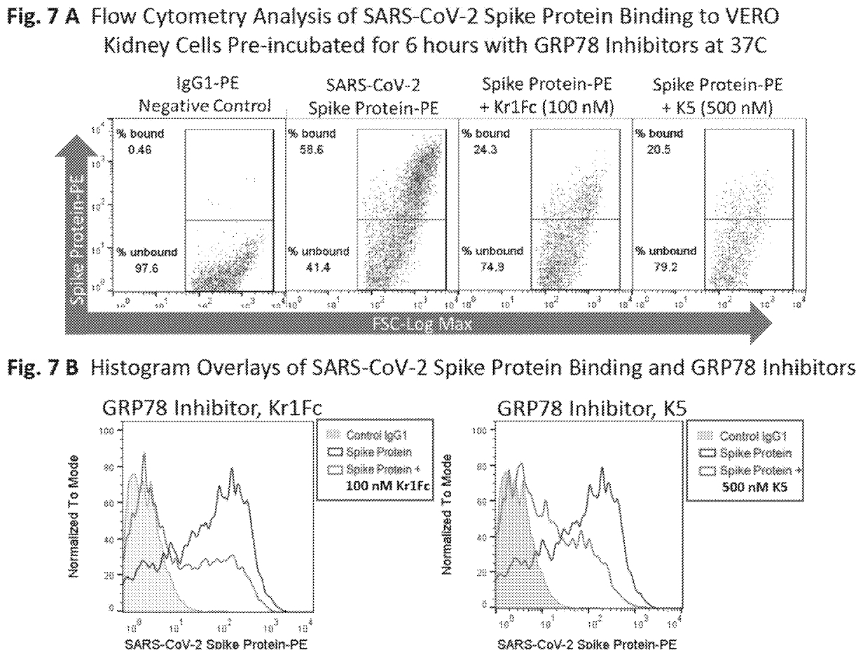

FIG. 7 shows that Kr1Fc and K5 prevent binding of SARS-CoV-2 spike protein either with preincubation with VERO cells or added at the same time as the spike protein (FIG. 6). FIG. 7A shows dot blot flow cytometry analysis and histogram overlay analysis of SARS-CoV-2 spike protein-PE (50 nM) binding to VERO cells preincubated with or without Kr1Fc (100 nM) or K5 (500 nM) for 6 hours. The inhibition of SARS-CoV-2 spike protein-PE binding was greater than 60% with Kr1 or K5 preincubation. These data suggest that preincubation of cells with GRP78 inhibitors may reduce spike protein binding slightly better.

FIG. 8 demonstrates that N-terminal GRP78 inhibitors reduce the expression of surface-bound GRP78 on VERO cells. To help define if our GRP78 inhibitors simply block the binding to SARS-CoV-2 spike protein to GRP78 or actually remove GRP78 from the cell surface, resulting in less surface-bound GRP78 for spike protein binding and internalization, we measured the amount of surface-bound GRP78 on VERO cells after Kr1Fc (100 nM) and K5 (500 nM) treatment. FIG. 8 A shows Flow cytometry analysis of VERO cells surface-bound GRP78 using a C-terminal GRP78 antibody-PE. This antibody has not been shown to inhibit SARS-CoV-2 spike protein binding. FIG. 8B shows the flow cytometry dot blot and histogram overlay analyses Kr1Fc and K5 produce a greater than 50% decrease in surface-bound GRP78. This suggests that these N-terminal GRP78 inhibitors may internalize/remove GRP78 from VERO cell surfaces resulting in less binding sites for SARS-CoV-2 spike protein binding.

FIG. 9 demonstrates that N-terminal GRP78 inhibitors reduce the expression of surface-bound GRP78 on A549 lung cells. To help define if our GRP78 inhibitors can internalize/remove GRP78 from A549 lung cell surfaces like VERO cells, we measured the amount of surface-bound GRP78 on A549 cells after Kr1Fc (100 nM) and K5 (500 nM) treatment. FIG. 9 A shows flow cytometry analysis of A549 cells surface-bound GRP78 shows a much lower expression of surface-bound GRP78 than VERO cells. Again, suggesting why VERO cells may be much more susceptible to infection than A549 cells. FIG. 9B shows the flow cytometry dot blot and histogram overlay analyses, Kr1Fc and K5 produce a decrease in surface-bound GRP78 from 84% to 60% respectfully. This suggests that these N-terminal GRP78 inhibitors may internalize/remove GRP78 from A540 and VERO cell surfaces resulting in less binding sites for SARS-CoV-2 spike protein binding.

FIG. 10 shows that surface-bound GRP78 expression on VERO cells, which is significantly reduced by Kr1Fc and K5, is essential for SARS-CoV-2 spike protein internalization. In FIG. 10 A, an assay, a pHrodo red dye was used to label SARS-CoV-2 spike protein to measure internalization. pHrodo dyes have a weak fluorescence at pH 7 (outside or bound to cells) but have a strong fluorescence inside the cell at a pH around 5.5 to 4.5. This allows for the measurement of internalization of the pHrodo red-labeled SARS-CoV-2 spike protein on VERO cells. FIG. 10B shows flow cytometry analysis of SARS-CoV-2 spike protein-pHrodo internalization on VERO cells with and without Kr1Fc and K5. From the dot blot analysis and the FIG. 10C histogram overlay plots, Kr1Fc (100 nM) and K5 (500 nM) significantly reduce the internalization of SARS-CoV-2 spike protein-pHrodo by over 95% in VERO cells.

FIG. 11 demonstrates that GRP78 inhibition with N-terminal GRP78 binding proteins, Kr1Fc and K5, potently and significantly inhibit whole SARS-CoV-2 pseudotyped virus attachment and internalization on VERO cells. In this invention, modified SARS-CoV-2 virus assay was performed by IBT Bioservices (Rockville, MD). In this assay, a SARS-CoV-2 pseudotyped virus was generated by replacing the replication RNA piece from the SARS-CoV-2 virus with RNA encoding the Luciferase enzyme protein for detection. The remaining structural proteins (spike protein, envelope protein, and matrix protein and nucleocapsid protein) were left intact. This allows for SARS-CoV-2 (rVSV-SARS-CoV-2 (D614G)) pseudotyped virus to attach and internalize but not replicate. The invention teaches that Kr1Fc and K5 can inhibit the full SARS-CoV-2 pseudotyped virus from attaching and entry into VERO cells. FIG. 11 A shows potent, dose dependent and complete neutralization (99.9%) of SARS-CoV-2 pseudotyped virus infectivity with IC₅₀ values of 3.5 uM for Kr1Fc, and 47.4 uM for K5 was observed. The assay was performed by using eight dilutions from 0.5 nM to 500 uM for K5 and 0.01 nM to 62 uM for Kr1Fc. These dilutions were added to triplicate wells in a 96 well plate with VERO cells (1×10⁵ cells per well). Wells in accordance with the invention are infected with SARS-CoV-2 pseudotyped virus at 25,000-35,000 Relative Light Units in each well. Plates were incubated for 24 hours, and attached cells were washed and then each well was read for Luciferase activity using Bright-Glo Assay System Kit (Promega). As shown in FIG. 11B the toxicity of the test compounds was also determined in parallel against VERO cells without virus. Kr1Fc displayed no cellular toxicity up to 2 mM and K5 inhibited VERO cells growth at a cytotoxic cell concentration at 50% (CC₅₀) of 375 uM. These toxicity numbers are at least 10-fold higher than the virus inhibition concentrations indicating toxicity is not the mechanism of action for virus attachment and entry inhibition.

FIGS. 12A and B illustrate K5 significantly augments co-inhibitory (PD-L1, B7H4) checkpoint protein and co-stimulatory (CD86, MHC-II) protein expressions induced by soluble GRP78 on A549 lung cells. Flow cytometry histogram plots of checkpoint protein expression +/− sGRP78 (5 ug/m) and K5 (500 nM). A549 cells were incubated for 3 days+/− sGRP78 (5 ug/ml) and K5 (500 nM). The cells were then fixed and stained with fluorescently labeled antibodies as indicated and FACs analysis was performed on a Guava PCA. All antibodies were mouse anti-human and PE labeled. A mouse IgG-PE antibody (grey) was used as a negative control. Average of 3 independent analyses with different A549 cell split numbers between 5-10 were used. ns: not significant, * p<0.05, ** p<0.01, ***p<0.005, ****p<0.001. Unless otherwise explained in context, asterisks “*” correspond to those in the figures.

FIGS. 13A and B show that Kr1Fc significantly augments expression of co-inhibitory (PD-L1, B7H4) checkpoint protein and co-stimulatory (CD86, MHC-II) protein expressions induced by soluble GRP78 on A549 lung cells. Flow cytometry histogram plots of checkpoint protein expression +/− sGRP78 (5 ug/m) and Kr1Fc (100 nM). A549 cells were incubated for 3 days +/− sGRP78 (5 ug/ml) and Kr1Fc (100 nM). The cells were then fixed and stained with fluorescently labeled antibodies as indicated and FACs analysis was performed on a Guava PCA. All antibodies were mouse anti-human and PE labeled. A mouse IgG-PE antibody (grey) was used as a negative control. Average of 3 independent analyses with different A549 cell split numbers between 5-10 were used. ns: not significant, * p<0.05, ** p<0.01, ***p<0.005, ****p<0.001

FIGS. 14A and B demonstrate that Kr1Fc and K5 inhibit soluble GRP78 induced cytokine expression of IL10 and IL6 from A549 lung cells. A549 cells (50,000) were added to Eppendorf tubes in full DMEM media. In half the tubes, soluble GRP78 (5 ug/ml) was added along with either K5 (500 nM) or Kr1Fc (100 nM). An IgG human antibody was used as a negative control. After incubation for 3 days at 37C with mild shaking, the cells were spun down and the supernatant was tested using either an IL10 or IL6 ELISA assay kits (R&D Systems). The protocol for each kit was followed and pg/ml of each cytokine for each condition for A549 cells with and with GRP78 inhibitors was calculated from standard curves. Each line is an average of 3 wells. ****p<0.001

FIG. 15 shows that Kr1Fc and K5 inhibit the activation of human plasminogen on VERO cell surfaces. In FIG. 15 A the schematic of undamaged lung on left side with effective and smooth blood flow. On right side is the damaged lung from SARS-CoV-2 virus infection. The infection causes intense inflammation, and hyperfibrinolysis inducing microthrombi and high levels of D-dimer. The red Plasminogen to plasmin reaction is where the GRP78 inhibitors block the formation of microthrombi and D-dimer by inhibiting cell surface plasmin activation. FIG. 15B shows significant inhibition of plasmin activation by Kr1 and K5 on VERO cells. In a 96 well plate, VERO cells were added (50,000 per well) and incubated overnight at 37C, 5% CO2. The next day the media was removed and PBS was added to each well. GRP78 inhibitors, Kr1Fc at (100 nM, 10 nM) and K5 at (500 nM, 50 nM) plus human plasminogen (100 ng) was then added to each well. Finally, plasmin substrate VAL-Leu-Lys-pNA (52251) was added to each well per manufactures instructions and the plate was read at 405 nm to measure pNA formation which is proportional to the enzymatic activity of plasmin. As shown in the chart, Kr1Fc and K5 significantly inhibited the Vmax for plasminogen activation on VERO cells.

FIG. 16 shows the mice treated with Kr1Fc (60 mg/kg) or K5 (90 mg/kg) every other day, intraperitoneally displayed no weight loses or overt toxicity. Kr1Fc and K5 were dosed in BALB/c mice 8 weeks old. Three groups of mice were dosed intraperitoneal (i.p.) in a volume of 10 mL/kg scaled to the body weight of each individual animal. Treatment groups were; 1 group treated PBS, i.p. every other day until day 22; 1 group treated with Kr1Fc at 60 mg/kg, i.p., every other day (qod) until day 22; and 1 group received K5 at 90 mg/kg every other day (qod) until day 22. Animals were weighed daily on Days 1-5, and then twice weekly until day 22. The mice were observed frequently for overt signs of any adverse, treatment-related (TR) side effects, and clinical signs were recorded when observed. Individual body weight was monitored as per protocol, and any animal with weight loss exceeding 30% for one measurement or exceeding 25% for three consecutive measurements was euthanized as a TR death. Group mean body weight loss was also monitored according to Charles River Discovery Services protocol. Acceptable toxicity was defined as a group mean body weight (BW) loss of less than 20% during the study and no more than 10% TR deaths. Deaths were classified as TR if it was attributable to treatment side effects as evidenced by clinical signs and/or necropsy. From FIG. 16, it is clear that Kr1Fc and K5 showed no overt toxicity or weight loss in mice dosed as listed above.

FIG. 17 is a table showing proposed GRP78 binding sequences from various viruses' protein domains compared to known GRP78 binding sequences from K5 (binding sequence reflected as SEQ ID NO: 190). This comparison of binding sequences identifies viruses that may be inhibited by our method of treatment which occurs through blocking the N-terminal domain of GRP78.

Treatment Parameters when Using the Therapeutic Invention

Severe viral illnesses are a result of exposure, cellular infection and replication of the virus to viral levels that overpower the host's immune system leading to signs and symptoms of disease. Viruses can't make new viruses on their own and require host cells to produce new viruses. With low levels of viral exposure, this typically results in the body's immune system to activate and attack the virus directly prior to symptom development. This results in the virus being eliminated from the host. In people with limited immune response and/or high levels of viral exposure, the host immune system will most likely become overpowered by the viral load and the patient will become ill. Viral infection propagates as the virus attaches to the cell surface at which time it is brought into the cell. Viruses bring in their own DNA or RNA instructions into the cell for replication and their eventual release only to repeat the process in greater numbers again and again increasing the infection rate, which increases the viral load and eventually leading to severe illness.

Viruses are known to have several cellular binding proteins called “spike proteins” on the surface which seek out and attach to the cell surface. For the SARS-CoV and SARS-CoV-2 viruses, this attachment can be to the ACE2 receptor and other accessory receptors like GRP78, which are found on the cell surface of respiratory lung and endothelial cells. These receptors then transport the viral material into the cells where it is replicated and expelled from the infected cells to repeat the process.

Method to Select and Inhibit Viruses with a GRP78 Recognition Site.

Surface bound GRP78 has been reported to be important for attachment and entry into host cells for several different types of viruses. In Elfiky's publication, they predict that coronaviruses SARS-CoV-2, NL63, 229E, OC43, and HKU1 have a similar GRP78 recognition/binding site. Other publications have shown that surface bound GRP78 is important for attachment and entry of Zika virus, Ebola virus, MERS-CoV, Coxsackievirus A9, Dengue virus, Japanese encephalitis virus (JEV) and Influenza viruses but have not examined if they have the same GRP78 binding sequences. From our previous publications, we demonstrated that the sequence from kringle 5 of human plasminogen, CYTTNPRKLYDYC binds tightly to surface bound GRP78.

Although, Elfiky's prediction uses a weaker GRP78 binding sequence, PEP42 (CTVALPGGYVRVC), than the K5 sequence, it still retains some of the important amino acids that we had shown previously for GRP78 binding in the kringle 5 structural fold region. In FIG. 17, we show sequence alignment of viruses compared to our known GRP78 binding kringle sequences to identify possible GRP78 binding sites. Using this alignment, we can predict which viruses will be responsive to our method of treatment for viral inhibition through blocking the N-terminal domain of GRP78. From this alignment, we can now predict that SARS-CoV-2, NL63, 229E, OC43, HKU1, MERS-CoV, EBOLA, Zika, Yellow Fever, West Nile, and Influenza A, B viruses will be inhibited by our therapies due to their GRP78 binding. However, SARS-CoV does not contain the proposed GRP78 binding sequence so we predict that our GRP78 inhibitors would not be effective against this virus. As proof this prediction is accurate, in Example 6, we can show that our N-terminal binding GRP78 inhibitor, Kr1Fc, potently blocks attachment and entry of SARS-CoV-2 pseudoviruses, but we have recently discovered that our GRP78 inhibitor, Kr1Fc, did not block viral induced death or weight loss in mice infected with a lethal variant of SARS-CoV.

Method for Selecting Our Target Population for Prophylactic or Therapeutic Treatment with GRP78 Inhibitors against SARS-CoV-2 and Other Viruses Infection

People under the age of 65 with comorbidities like cancer, obesity, cardiac disease, lung disease, and hypertension, have a higher risk of being infected with SARS-CoV-2 virus than those without a comorbidity. This same group of people also has a much higher rate of mortality from COVID19. For people over 65 with or without a comorbidity their chances of dying from COVID19 are also much higher than those under the age of 65. Due to stress applied to endothelial, lung, respiratory and immune cells by these comorbidities and in general the aging process, the presence of surface bound GRP78 has recently been recognized as an important player in aging and disease progression.

With aging and with comorbidities, cells become more stressed leading to consistent and higher expression of cell surface bound GRP78. This higher expression of the GRP78 allows for more viral binding and entry into cells (FIG. 1). Viral replication and release of the viral particles can then occur at a much higher rate. This process of increased binding, entry and replication of viruses due to increased GRP78 receptor can then overpower the immune system leading to increased morbidity and mortality.

Treating Patients with Autoimmune Diseases

Patients with autoimmune diseases do not have a fully functioning immune system and have been reported to present with higher expression of surface bound GRP78. Current treatments and vaccines that are designed to stimulate the immune system against viruses like SARS-CoV-2 are much less effective in these patients that are prone to unwanted increased viral progression. Providing prophylactic and/or therapeutic support with our GRP78 inhibitors to these patients may provide a level of protection against infection and if infected will block new viral attachment, entry, replication and release of new viral particles into the patient.

Treating Patients with Cancer

Patients with cancer, especially lung and blood borne cancers, have been shown to have a higher expression of surface bound GRP78 on their lower and higher respiratory tract cells as well as their endothelial cells. These patients, because of their chemotherapy treatments, usually have a reduced immune response to vaccines and pathogens. By treating these patients, either prophylactically or after infection SARS-CoV-2 virus, with our GRP78 inhibitions may allow for a decreased chance for infection and a decreased rate of viral infectivity by blocking virus access to the surface bound GRP78 receptor.

Treating Patients with Obesity.

Patients with obesity have several comorbidities like hypertension, diabetes, and poor circulation which can cause stressed lung and endothelial cells resulting in increased surface expressed GRP78. This increase in surface expressed GRP78 makes this group of people at high risk for SARS-CoV-2 infection and death. Using our GRP78 inhibitors to block the amount of viral infectivity through surface bound GRP78, we may greatly reduce symptoms and the severity of the viral infection.

Treating Patients with Diabetes

Patients with diabetic type 1 or type 2 disease have been shown to have higher expression of surface bound GRP78 on kidney and endothelial cells. With the use of our GRP78 inhibitors in these patients, the viral load and chance of SARS-CoV-2 infection should be greatly reduced allowing for normal immune attack and elimination of viral particles.

Treating Patients with Cardiovascular Disease

A hallmark for patients with cardiovascular disease is increased endoplasmic stress in heart tissues leading to higher expression of ACE2 and surface bound GRP78 on their vasculature cells. Although, increased ACE2 expression allows for increased SARS-CoV-2 attachment, this invention teaches that SARS-CoV-2 requires surface bound GRP78 for viral entry and as such, by reducing the surface expression of GRP78 with our GRP78 inhibitors, this should lead to reduced viral infection and mortality.

Treating Patients with Hypertension

Studies show that patients with hypertension have increased endoplasmic reticulum expressed GRP78 in their arterial cells. This increase in GRP78 expression leads to an increase in surface bound GRP78. As listed above, surface expression of GRP78 allows for higher SARS-CoV-2 viral attachment, entry and replication in cells. By reducing the expression of surface bound GRP78 with our inhibitors, not only will they reduce the risk for viral infection but also reduce chronic hypertension effects.

### Treating Patients Over 65 Years of Age

With aging, inflammation is more prevalent, which leads to muscle and arterial cells being more stressed resulting in consistent expression of surface bound GRP78. It is clear that SARS-CoV-2 infection severity increases substantially with age. This higher level of GRP78 receptor allows for availability of more attachment and entry of SARS-CoV-2 virus into the cells. The treatment of patients over the age of 65 with GRP78 inhibitors will lead to reduced surface bound GRP78 on stressed unhealthy cells. With less receptors for SARS-CoV-2 virus attachment, the less severe the SARS-CoV-2 infection will be.

It is rare that young (<60), heathy people show severe symptoms from SARS-CoV-2 infection. We believe this is due to the fact that surface bound GRP78 on young, heathy people's cells is very low, which decreases the risk of viral attachment. This limits the severity of infection and greatly decreases the mortality rate of children and younger people. As such, there is no need to treat this population with our GRP78 inhibitors.

However, there are exceptions to this theory. For example, Influenza viruses infect the young and the old even though the viruses contain a GRP78 binding site and there is no surface bound GRP78 on their cells. Recent publications show that infected cells induce surface GRP78 expression, which leads to more infection. In these cases, treatment with GRP78 inhibitors will reduce surface bound GRP78, resulting in decreased viral load and a reduced time of infectivity.

### Types of Treatment

For our GRP78 inhibitors, this invention shows two type of treatment options. The first is to give our GRP78 inhibitors as a treatment for patients listed above after SARS-CoV-2 infection. Infected patients with comorbidities will benefit from decreased surface bound GRP78 resulting in decreased virus attachment and entry.

The second method to use for our GRP78 inhibitors is to give them prophylactically. For the patients listed above with comorbidities and for those above 65 years of age whose lung, respiratory and arterial cells have increased surface bound GRP78 expression, treatment with our GRP78 inhibitors prophylactically will reduce the risk of SARS-CoV-2 viral infection and disease progression. Our GRP78 inhibitors given prophylactically will may also be effective at protecting health care workers, first responders, people who travel to high infection areas and teachers from SARS-CoV-2 infection.

### Co-Treatment Options

This invention shows that our GRP78 inhibitors may be given in combination with other types of therapies to help reduce viral infection and the resulting symptoms. For example:
anti-inflammatory agents like Dexamethasone have been shown to reduce the symptoms like cytokine inflammation leading to increased survival,
anti-viral agents like Remdesivir which has been shown to block replication of viruses leading to reduced viral disease.
immune suppressive agents like Baricitinib (JAK inhibitor), and Tocilzumab (IL6 inhibitor) which have not been shown to be effective in late-stage disease but show promise in early to mid-stage disease.
anti-coagulant agents like heparin which significantly reduced the symptoms of clot storms as a result of SARS-CoV-2 infection.
vaccines like those against SARS-CoV-2, or influenza may be effective against the main strain but not against evolving variants. By using both vaccines and GRP78 inhibitors, they may be more effective for cures.
   Problems with Current Therapies and Vaccines Against SARS-CoV-2 Virus:

Vaccines against the SARS-CoV-2 virus are designed to force the immune system to attack the virus through either the spike protein on the virus surface or other envelope proteins. This then allows the body to make antibodies against the SARS-CoV-2 virus spike protein, which block SARS-CoV-2 virus attaching and internalizing into the host cell. Currently, there are three approved vaccines against the spike protein of SARS-CoV-2 virus. For people with a weaken immune system due to comorbidities or other drug treatments, these vaccines may not as effective. Also SARS-CoV-2 variant (B.1.351) infections have been shown to be resistant to the AstraZeneca Covid-19 vaccine for mild-to-moderate infections. This is the underlying issue with anti-viral vaccines in that there is a need for yearly updated versions of the vaccine to combat evolving variants.

Monoclonal antibodies against SARS-CoV-2 spike protein have been approved for emergency use by the FDA. These antibodies have been shown to be effective against patients with early SARS-CoV-2 infections. Patients that were hospitalized did not show any improvement. Finally, like vaccines these therapies are very targeted against the viral spike protein and variants may not be inhibited.

Remdesivir is a protease inhibitor that has been approved for use by the FDA. It also failed against hospitalized patients and did not prevent death. If give early in the SARS-CoV-2 viral infection, it was shown to decrease hospitalization time and duration of the infection.

Listed above are the only FDA approved therapies for COVID19 disease as of the writing of this invention. However, several others are progressing through clinical studies that show some promise. Baricitinib is a Janus Kinase inhibitor that is an immunosuppressant. This therapy has shown to be effective against SARS-CoV-2 virus infected patients on oxygen but not on ventilation. Again, late-stage COVID19 disease is not addressed. Tocilizumab is a monoclonal antibody against IL-6. Inhibiting IL-6 treats SARS-CoV-2 infection symptoms by tamping down inflammation allowing the immune system to function better. The outcomes from late-stage patients receiving Tocilzumab were reduced time to discharge for both people on ventilation and oxygen.

This invention teaches a method for treating patients with SARS-CoV-2 infection that uses N-terminal GRP78 inhibitors. Unlike the vaccines or current viral treatments, our therapy addresses the potential viral infection at the cell surface, not at the virus surface. By inhibiting the host cells receptors and not the virus, mutant variants will not be resistant to GRP78 inhibitor therapy. Also, since GRP78 inhibitors do not rely on the immune system for viral clearance or inhibition, immune suppressed patients can still be treated. As such, late stage COVID19 diseased patients, patients with suppressed immune systems or patients with comorbidities will still be sensitive to our GRP78 inhibitors, which will reduce infection time leading to less hospital stay and viral load.

FIG. 17 is a table showing protein sequence comparisons for GRP78 binding sites of K5 (SEQ ID NO: 190) and selected peptide sequences from various viruses. Amino acids underlined twice in FIG. 17 are identical to K5 (SEQ ID NO: 190) GRP78 binding site amino acids, and amino acids that are similar in type to those of K5 (SEQ ID NO: 190) are underlined only once. Where there is no underline under the amino acid, it is concluded there is no similarity. An asterisk (*) in FIG. 17 marks the amino acids important for GRP78 binding. The Cysteine amino acid at the beginning of the GRP78 binding sites is found in almost all sequences.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of such claims as shall be appended.

In accordance with my invention, I claim:

1. A method for the treatment comprising administering to a person in need an effective dose of the antivirus agent of claim 1 for prevention (prophylactic) and inhibition (treatment) of viral infection for viruses that use surface-bound GRP78 for attachment, entry and replication like SARS-CoV-2 virus, SARS-CoV virus, MER-CoV virus, Japanese Encephalitis Virus, Coxsackievirus, Dengue virus and Influenza A&B viruses.

2. The antivirus agent of claim 1, comprising:
a GRP78 antagonist or a pharmaceutically acceptable salt:
wherein the GRP78 antagonist is selected from a group consisting of a plasminogen kringle 5 fragment, a plasminogen kringle 5 fragment attached to immunoglobulin, an ROR1 kringle fragment, an ROR1 kringle fragment attached to an immunoglobulin, an ROR2 kringle fragment, and an ROR2 kringle fragment attached to an immunoglobulin.

3. The antivirus agent of claim 2, wherein the plasminogen kringle 5 fragment is of SEQ ID NO:1 and combinations thereof.

4. The antivirus agent of claim 2, wherein the plasminogen kringle 5 fragment attached to immunoglobulin is selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and combinations thereof.

5. The antivirus agent of claim 2, wherein the ROR1 kringle fragment is of SEQ ID NO:19, and combinations thereof.

6. The antivirus agent of claim 2, wherein the ROR1 kringle fragment attached to immunoglobulin is selected from the group consisting of SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, and combinations thereof.

7. The antivirus agent of claim 2, wherein the ROR2 kringle fragment is SEQ ID NO:33 and combinations thereof.

8. The antivirus agent of claim 2, wherein the ROR2 kringle fragment attached to immunoglobulin is selected from the group consisting of SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, and combinations thereof.

9. The antivirus agent of claim 2, wherein the GRP78 antagonist binds to the N-terminal GRP78 domain and competes with claim 1 GRP78 antagonists to induce GRP78 internalization.

10. An antivirus agent comprising:
a GRP78 antagonist or a pharmaceutically acceptable salt:
wherein the GRP78 antagonist is selected from a group consisting of a plasminogen kringle 5 fragment, a plasminogen kringle 5 fragment attached to immunoglobulin, an ROR1 kringle fragment, an ROR1 kringle fragment attached to an immunoglobulin, an ROR2 kringle fragment, and an ROR2 kringle fragment attached to an immunoglobulin.

11. The antivirus agent of claim 10, wherein the plasminogen kringle 5 fragment is of SEQ ID NO:1 and combinations thereof.

12. The antivirus agent of claim 10, wherein the plasminogen kringle 5 fragment attached to immunoglobulin is selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and combinations thereof.

13. The antivirus agent of claim 10, wherein the ROR1 kringle fragment is of SEQ ID NO:19, and combinations thereof.

14. The antivirus agent of claim 10, wherein the ROR1 kringle fragment attached to immunoglobulin is selected from the group consisting of SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, and combinations thereof.

15. The antivirus agent of claim 10, wherein the ROR2 kringle fragment is SEQ ID NO:33 and combinations thereof.

16. The antivirus agent of claim 10, wherein the ROR2 kringle fragment attached to immunoglobulin is selected from the group consisting of SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, and combinations thereof.

17. The antivirus agent of claim 10, wherein the GRP78 antagonist binds to the N-terminal GRP78 domain and competes with claim 1 GRP78 antagonists to induce GRP78 internalization.


SEQUENCE LISTING (189 sequences)

SEQ ID NO: 1 - protein, 89 aa, synthetic construct
  REGION 1..89: note = Synthetic Peptide K5(3-89 Active Zone)
SEEDCMFGNGKGYRGKRATTVTGTPCQDWAAQEPHRHSIFTPETNPRAGLEKNYCRNPDGDVGGPWCYTTNPRKLYDYCDVPQCAAPKS

SEQ ID NO: 2 - protein, 317 aa, synthetic construct
  REGION 1..317: note = Synthetic Peptide K5(3-89 Active Zone)frag-Fc
SEEDCMFGNGKGYRGKRATTVTGTPCQDWAAQEPHRHSIFTPETNPRAGLEKNYCRNPDGDVGGPWCYTTNPRKLYDYCDVPQCAAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 254 aa, synthetic construct
  REGION 1..254: note = Synthetic Peptide K5(57-81 Active Zone)frag -Fc
RNPDGDVGGPWCYTTNPRKLYDYCKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 254 aa, synthetic construct
  REGION 1..254: note = Synthetic Peptide K5(57-81 Active Zone w Ala sub Cys)frag -Fc
RNPDGDVGGPWAYTTNPRKLYDYAKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 254 aa, synthetic construct
  REGION 1..254: note = Synthetic Peptide K5(57-81 Active Zone w Val sub Cys)frag-Fc
RNPDGDVGGPWVYTTNPRKLYDYVKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 254 aa, synthetic construct
  REGION 1..254: note = Synthetic Peptide K5(57-81 Active Zone w Ile sub Cys)frag-Fc
RNPDGDVGGPWIYTTNPRKLYDYIKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 254 aa, synthetic construct
  REGION 1..254: note = Synthetic Peptide K5(57-81 Active Zone w Leu sub Cys)frag-Fc
RNPDGDVGGPWLYTTNPRKLYDYLKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 250 aa, synthetic construct
  REGION 1..250: note = Synthetic Peptide K5(70-89 Active Zone)frag-Fc
YTTNPRKLYDYCDVPQCAAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 250 aa, synthetic construct
  REGION 1..250: note = Synthetic Peptide K5(70-89 Active Zone with Ala sub Cys)frag-Fc
YTTNPRKLYDYADVPQAAAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 250 aa, synthetic construct
  REGION 1..250: note = Synthetic Peptide K5(70-89 Active Zone with Val sub Cys)frag-Fc
YTTNPRKLYDYVDVPQVAAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 250 aa, synthetic construct
  REGION 1..250: note = Synthetic Peptide K5(70-89 Active Zone with Ile sub Cys)frag-Fc
YTTNPRKLYDYIDVPQIAAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 250 aa, synthetic construct
  REGION 1..250: note = Synthetic Peptide K5(70-89 Active Zone with Leu sub Cys)frag-Fc
YTTNPRKLYDYLDVPQLAAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 246 aa, synthetic construct
  REGION 1..246: note = Synthetic Peptide K5(74-89 Active Zone)frag-Fc
PRKLYDYCDVPQCAAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 246 aa, synthetic construct
  REGION 1..246: note = Synthetic Peptide K5(74-89 Active Zone w Ala sub Cys)frag-Fc
PRKLYDYADVPQAAAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 246 aa, synthetic construct
  REGION 1..246: note = Synthetic Peptide K5(74-89 Active Zone w Val sub Cys)frag-Fc
PRKLYDYVDVPQVAAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 246 aa, synthetic construct
  REGION 1..246: note = Synthetic Peptide K5(74-89 Active Zone w Ile sub Cys)frag-Fc
PRKLYDYIDVPQIAAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 246 aa, synthetic construct
  REGION 1..246: note = Synthetic Peptide K5(74-89 Active Zone w Leu sub Cys)frag-Fc
PRKLYDYLDVPQLAAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 237 aa, synthetic construct
  REGION 1..237: note = Synthetic Peptide K5(74-80 Active Zone)frag-Fc
PRKLYDYKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 93 aa, synthetic construct
  REGION 1..93: note = Synthetic Peptide Kr1(1-93 Active Zone)frag
GSNKNHKCYNSTGVDYRGTVSVTKSGRQCQPWNSQYPHTHTFTALRFPELNGGHSYCRNPGNQKEAPWCFTLDENFKSDLCDIPACDSKDSDI

SEQ ID NO: 20 - protein, 317 aa, synthetic construct
  REGION 1..317: note = Synthetic Peptide Kr1(3-91 Active Zone)frag-Fc
NKNHKCYNSTGVDYRGTVSVTKSGRQCQPWNSQYPHTHTFTALRFPELNGGHSYCRNPGNQKEAPWCFTLDENFKSDLCDIPACDSKDSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 262 aa, synthetic construct
  REGION 1..262: note = Synthetic Peptide Kr1(58-91 Active Zone)frag-Fc
RNPGNQKEAPWCFTLDENFKSDLCDIPACDSKDSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 262 aa, synthetic construct
  REGION 1..262: note = Synthetic Peptide Kr1(58-91 Active Zone with Ala sub Cys)frag-Fc
RNPGNQKEAPWAFTLDENFKSDLADIPAADSKDSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 262 aa, synthetic construct
  REGION 1..262: note = Synthetic Peptide Kr1(58-91 Active Zone with Val sub Cys)frag-Fc
RNPGNQKEAPWVFTLDENFKSDLVDIPAVDSKDSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 262 aa, synthetic construct
  REGION 1..262: note = Synthetic Peptide Kr1(58-91 Active Zone with Ile sub Cys)frag-Fc
RNPGNQKEAPWIFTLDENFKSDLIDIPAIDSKDSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 262 aa, synthetic construct
  REGION 1..262: note = Synthetic Peptide Kr1(58-91 Active Zone with Leu sub Cys)frag-Fc
RNPGNQKEAPWLFTLDENFKSDLLDIPALDSKDSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 251 aa, synthetic construct
  REGION 1..251: note = Synthetic Peptide Kr1(58-80 Active Zone)frag-Fc
RNPGNQKEAPWCFTLDENFKSDLCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 251 aa, synthetic construct
  REGION 1..251: note = Synthetic Peptide Kr1(58-80 Active Zone w Ala sub Cys)frag-Fc
RNPGNQKEAPWAFTLDENFKSDLCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 251 aa, synthetic construct
  REGION 1..251: note = Synthetic Peptide Kr1(58-80 Active Zone w Val sub Cys)frag-Fc
RNPGNQKEAPWVFTLDENFKSDLCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 251 aa, synthetic construct
  REGION 1..251: note = Synthetic Peptide Kr1(58-80 Active Zone w Ile sub Cys)frag-Fc
RNPGNQKEAPWIFTLDENFKSDLCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 251 aa, synthetic construct
  REGION 1..251: note = Synthetic Peptide Kr1(58-80 Active Zone w Leu sub Cys)frag-Fc
RNPGNQKEAPWLFTLDENFKSDLCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 239 aa, synthetic construct
  REGION 1..239: note = Synthetic Peptide Kr1(70-80 Active Zone)frag-Fc
FTLDENFKSDLCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 236 aa, synthetic construct
  REGION 1..236: note = Synthetic Peptide Kr1(73-80 Active Zone)frag-Fc
DENFKSDLCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 85 aa, synthetic construct
  REGION 1..85: note = Synthetic Peptide Kr2(1-85 Active Zone)frag
GRYHQCYNGSGMDYRGTASTTKSGHQCQPWALQHPHSHHLSSTDFPELGGGHAYCRNPGGQMEGPWCFTQNKNVRMELCDVPSCS

SEQ ID NO: 34 - protein, 316 aa, synthetic construct
  REGION 1..316: note = Synthetic Peptide Kr2(1-85 Active Zone)frag-Fc
GRYHQCYNGSGMDYRGTASTTKSGHQCQPWALQHPHSHHLSSTDFPELGGGHAYCRNPGGQMEGPWCFTQNKNVRMELCDVPSCSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 261 aa, synthetic construct
  REGION 1..261: note = Synthetic Peptide Kr2(56-85 Active Zone)frag-Fc
RNPGGQMEGPWCFTQNKNVRMELCDVPSCSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 261 aa, synthetic construct
  REGION 1..261: note = Synthetic Peptide Kr2(56-85 Active Zone w Ala sub Cys)frag-Fc
RNPGGQMEGPWAFTQNKNVRMELADVPSASPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 261 aa, synthetic construct
  REGION 1..261: note = Synthetic Peptide Kr2(56-85 Active Zone w Val sub Cys)frag-Fc
RNPGGQMEGPWVFTQNKNVRMELVDVPSVSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 261 aa, synthetic construct
  REGION 1..261: note = Synthetic Peptide Kr2(56-85 Active Zone w Ile sub Cys)frag-Fc
RNPGGQMEGPWIFTQNKNVRMELIDVPSISPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 261 aa, synthetic construct
  REGION 1..261: note = Synthetic Peptide Kr2(56-85 Active Zone w Leu sub Cys)frag-Fc
RNPGGQMEGPWLFTQNKNVRMELLDVPSLSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 249 aa, synthetic construct
  REGION 1..249: note = Synthetic Peptide Kr2(68-85 Active Zone)frag-Fc
FTQNKNVRMELCDVPSCSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 249 aa, synthetic construct
  REGION 1..249: note = Synthetic Peptide Kr2(68-85 Active Zone w Ala sub Cys)frag-Fc
FTQNKNVRMELADVPSASPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 249 aa, synthetic construct
  REGION 1..249: note = Synthetic Peptide Kr2(68-85 Active Zone w Val sub Cys)frag-Fc
FTQNKNVRMELVDVPSVSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 249 aa, synthetic construct
  REGION 1..249: note = Synthetic Peptide Kr2(68-85 Active Zone w Ile sub Cys)frag-Fc
FTQNKNVRMELIDVPSISPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 249 aa, synthetic construct
  REGION 1..249: note = Synthetic Peptide Kr2(68-85 Active Zone w Leu sub Cys)frag-Fc
FTQNKNVRMELLDVPSLSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 241 aa, synthetic construct
  REGION 1..241: note = Synthetic Peptide Kr2(68-79 Active Zone w Lys sub Cys)frag-Fc
FTQNKNVRMELKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 24 aa, synthetic construct
  REGION 1..24: note = Synthetic Peptide Kr1(58-81 Active Zone w Ala sub Cys and w Lys sub Cys)frag
RNPGNQKEAPWAFTLDENFKSDLK

SEQ ID NO: 47 - protein, 24 aa, synthetic construct
  REGION 1..24: note = Synthetic Peptide Kr1(58-81 Active Zone w Val sub Cys and w Lys sub Cys)frag
RNPGNQKEAPWVFTLDENFKSDLK

SEQ ID NO: 48 - protein, 24 aa, synthetic construct
  REGION 1..24: note = Synthetic Peptide Kr1(58-81 Active Zone w Ile sub Cys and w Lys sub Cys)frag
RNPGNQKEAPWIFTLDENFKSDLK

SEQ ID NO: 49 - protein, 24 aa, synthetic construct
  REGION 1..24: note = Synthetic Peptide Kr1(58-81 Active Zone w Leu sub Cys and w Lys sub Cys)frag
RNPGNQKEAPWLFTLDENFKSDLK

SEQ ID NO: 50 - protein, 23 aa, synthetic construct
  REGION 1..23: note = Synthetic Peptide Kr1(58-80 Active Zone w Ala sub Cys)frag
RNPGNQKEAPWAFTLDENFKSDL

SEQ ID NO: 51 - protein, 23 aa, synthetic construct
  REGION 1..23: note = Synthetic Peptide Kr1(58-80 Active Zone w Val sub Cys)frag
RNPGNQKEAPWVFTLDENFKSDL

SEQ ID NO: 52 - protein, 23 aa, synthetic construct
  REGION 1..23: note = Synthetic Peptide Kr1(58-80 Active Zone w Ile sub Cys)frag
RNPGNQKEAPWIFTLDENFKSDL

SEQ ID NO: 53 - protein, 23 aa, synthetic construct
  REGION 1..23: note = Synthetic Peptide Kr1(58-80 Active Zone w Leu sub Cys)frag
RNPGNQKEAPWLFTLDENFKSDL

SEQ ID NO: 54 - protein, 12 aa, synthetic construct
  REGION 1..12: note = Synthetic Peptide Kr1(58-69 Active Zone w Lys sub Cys)frag
RNPGNQKEAPWK

SEQ ID NO: 55 - protein, 11 aa, synthetic construct
  REGION 1..11: note = Synthetic Peptide Kr1(58-68 Active Zone)frag
RNPGNQKEAPW

SEQ ID NO: 56 - protein, 12 aa, synthetic construct
  REGION 1..12: note = Synthetic Peptide Kr1(70-81 Active Zone w Lys sub Cys)frag
FTLDENFKSDLK

SEQ ID NO: 57 - protein, 11 aa, synthetic construct
  REGION 1..11: note = Synthetic Peptide Kr1(70-80 Active Zone)frag
FTLDENFKSDL

SEQ ID NO: 58 - protein, 10 aa, synthetic construct
  REGION 1..10: note = Synthetic Peptide Kr1(71-80 Active Zone)frag
TLDENFKSDL

SEQ ID NO: 59 - protein, 9 aa, synthetic construct
  REGION 1..9: note = Synthetic Peptide Kr1(72-80 Active Zone)frag
LDENFKSDL

SEQ ID NO: 60 - protein, 8 aa, synthetic construct
  REGION 1..8: note = Synthetic Peptide Kr1(73-80 Active Zone)frag
DENFKSDL

SEQ ID NO: 61 - protein, 7 aa, synthetic construct
  REGION 1..7: note = Synthetic Peptide Kr1(73-79 Active Zone)frag
DENFKSD

SEQ ID NO: 62 - protein, 79 aa, synthetic construct
  REGION 1..79: note = Synthetic Peptide Kr2(6-84 Active Zone)frag
CYNGSGMDYRGTASTTKSGHQCQPWALQHPHSHHLSSTDFPELGGGHAYCRNPGGQMEGPWCFTQNKNVRMELCDVPSC

SEQ ID NO: 63 - protein, 24 aa, synthetic construct
  REGION 1..24: note = Synthetic Peptide Kr2(56-79 Active Zone w Ala sub Cys and Lys sub Cys)frag
RNPGGQMEGPWAFTQNKNVRMELK

SEQ ID NO: 64 - protein, 24 aa, synthetic construct
  REGION 1..24: note = Synthetic Peptide Kr2(56-79 Active Zone w Val sub Cys and Lys sub Cys)frag
RNPGGQMEGPWVFTQNKNVRMELK

SEQ ID NO: 65 - protein, 24 aa, synthetic construct
  REGION 1..24: note = Synthetic Peptide Kr2(56-79 Active Zone w Ile sub Cys and Lys sub Cys)
RNPGGQMEGPWIFTQNKNVRMELK

SEQ ID NO: 66 - protein, 24 aa, synthetic construct
  REGION 1..24: note = Synthetic Peptide Kr2(56-79 Active Zone w Leu sub Cys and Lys sub Cys)frag
RNPGGQMEGPWLFTQNKNVRMELK

SEQ ID NO: 67 - protein, 23 aa, synthetic construct
  REGION 1..23: note = Synthetic Peptide Kr2(56-78 Active Zone w Ala sub Cys)frag
RNPGGQMEGPWAFTQNKNVRMEL

SEQ ID NO: 68 - protein, 23 aa, synthetic construct
  REGION 1..23: note = Synthetic Peptide Kr2(56-78 Active Zone w Val sub Cys)frag
RNPGGQMEGPWVFTQNKNVRMEL

SEQ ID NO: 69 - protein, 23 aa, synthetic construct
  REGION 1..23: note = Synthetic Peptide Kr2(56-78 Active Zone w Ile sub Cys)frag
RNPGGQMEGPWIFTQNKNVRMEL

SEQ ID NO: 70 - protein, 23 aa, synthetic construct
  REGION 1..23: note = Synthetic Peptide Kr2(56-78 Active Zone w Leu sub Cys)frag
RNPGGQMEGPWLFTQNKNVRMEL

SEQ ID NO: 71 - protein, 12 aa, synthetic construct
  REGION 1..12: note = Synthetic Peptide Kr2(56-67 Active Zone with Lys sub Cys)frag
RNPGGQMEGPWK

SEQ ID NO: 72 - protein, 11 aa, synthetic construct
  REGION 1..11: note = Synthetic Peptide Kr2(56-66 Active Zone)frag
RNPGGQMEGPW

SEQ ID NO: 73 - protein, 12 aa, synthetic construct
  REGION 1..12: note = Synthetic Peptide Kr2(68-79 Active Zone w Lys sub Cys)frag
FTQNKNVRMELK

SEQ ID NO: 74 - protein, 11 aa, synthetic construct
  REGION 1..11: note = Synthetic Peptide Kr2(68-78 Active Zone)frag
FTQNKNVRMEL

SEQ ID NO: 75 - protein, 10 aa, synthetic construct
  REGION 1..10: note = Synthetic Peptide Kr2(69-78 Active Zone)frag
TQNKNVRMEL

SEQ ID NO: 76 - protein, 9 aa, synthetic construct
  REGION 1..9: note = Synthetic Peptide Kr2(70-78 Active Zone)frag
QNKNVRMEL

SEQ ID NO: 77 - protein, 8 aa, synthetic construct
  REGION 1..8: note = Synthetic Peptide Kr2(70-77 Active Zone)frag
QNKNVRME

SEQ ID NO: 78 - protein, 24 aa, synthetic construct
  REGION 1..24: note = Synthetic Peptide Mod-Kr1(58-81 Active Zone w Ala sub Cys and w Lys sub Cys)frag
  VARIANT 1: note = Xaa is Arg linked to NAc
XNPGNQKEAPWAFTLDENFKSDLK

SEQ ID NO: 79 - protein, 24 aa, synthetic construct
  REGION 1..24: note = Synthetic Peptide Mod-Kr1(58-81 Active Zone w Val sub Cys and w Lys sub Cys)frag
  VARIANT 1: note = Xaa is Arg linked to NAc
XNPGNQKEAPWVFTLDENFKSDLK

SEQ ID NO: 80 - protein, 24 aa, synthetic construct
  REGION 1..24: note = Synthetic Peptide Mod-Kr1(58-81 Active Zone w Ile sub Cys and w Lys sub Cys)frag
  VARIANT 1: note = Xaa is Arg linked to NAc
XNPGNQKEAPWIFTLDENFKSDLK

SEQ ID NO: 81 - protein, 24 aa, synthetic construct
  REGION 1..24: note = Synthetic Peptide Mod-Kr1(58-81 Active Zone w Leu sub Cys and w Lys sub Cys)frag
  VARIANT 1: note = Xaa is Arg linked to NAc
XNPGNQKEAPWLFTLDENFKSDLK

SEQ ID NO: 82 - protein, 24 aa, synthetic construct
  REGION 1..24: note = Synthetic Peptide Mod-Kr1(58-81 Active Zone w Ala sub Cys and w Lys sub Cys)frag
  VARIANT 1: note = Xaa is Arg linked to MPA
XNPGNQKEAPWAFTLDENFKSDLK

SEQ ID NO: 83 - protein, 24 aa, synthetic construct
  REGION 1..24: note = Synthetic Peptide Mod-Kr1(58-81 Active Zone w Val sub Cys and w Lys sub Cys)frag
  VARIANT 1: note = Xaa is Arg linked to MPA
XNPGNQKEAPWVFTLDENFKSDLK

SEQ ID NO: 84 - protein, 24 aa, synthetic construct
  REGION 1..24: note = Synthetic Peptide Mod-Kr1(58-81 Active Zone w Ile sub Cys and w Lys sub Cys)frag
  VARIANT 1: note = Xaa is Arg linked to MPA
XNPGNQKEAPWIFTLDENFKSDLK

SEQ ID NO: 85 - protein, 24 aa, synthetic construct
  REGION 1..24: note = Synthetic Peptide Mod-Kr1(58-81 Active Zone w Leu sub Cys and w Lys sub Cys)frag
  VARIANT 1: note = Xaa is Arg linked to MPA
XNPGNQKEAPWLFTLDENFKSDLK

SEQ ID NO: 86 - protein, 24 aa, synthetic construct
  REGION 1..24: note = Synthetic Peptide Mod-Kr1(58-81 Active Zone w Ala sub Cys and w Lys sub Cys)frag
  VARIANT 1: note = Xaa is Arg linked to MPA-AEEA
XNPGNQKEAPWAFTLDENFKSDLK

SEQ ID NO: 87 - protein, 24 aa, synthetic construct
  REGION 1..24: note = Synthetic Peptide Mod-Kr1(58-81 Active Zone w Val sub Cys and w Lys sub Cys)frag
  VARIANT 1: note = Xaa is Arg linked to MPA-AEEA
XNPGNQKEAPWVFTLDENFKSDLK

SEQ ID NO: 88 - protein, 24 aa, synthetic construct
  REGION 1..24: note = Synthetic Peptide Mod-Kr1(58-81 Active Zone w Ile sub Cys and w Lys sub Cys)frag
  VARIANT 1: note = Xaa is Arg linked to MPA-AEEA
XNPGNQKEAPWIFTLDENFKSDLK

SEQ ID NO: 89 - protein, 24 aa, synthetic construct
  REGION 1..24: note = Synthetic Peptide Mod-Kr1(58-81 Active Zone w Leu sub Cys and w Lys sub Cys)frag
  VARIANT 1: note = Xaa is Arg linked to MPA-AEEA
XNPGNQKEAPWLFTLDENFKSDLK

SEQ ID NO: 90 - protein, 23 aa, synthetic construct
  REGION 1..23: note = Synthetic Peptide Mod-Kr1(58-81 Active Zone w Ala sub Cys and w Lys sub Cys)frag
  VARIANT 1: note = Xaa is Arg linked to NAc
  VARIANT 23: note = Xaa is Lys linked to N-epsilon-MPA
XPGNQKEAPWAFTLDENFKSDLX

SEQ ID NO: 91 - protein, 23 aa, synthetic construct
  REGION 1..23: note = Synthetic Peptide Mod-Kr1(58-81 Active Zone w Val sub Cys and w Lys sub Cys)frag
  VARIANT 1: note = Xaa is Arg linked to NAc
  VARIANT 23: note = Xaa is Lys linked to N-epsilon-MPA
XPGNQKEAPWVFTLDENFKSDLX

SEQ ID NO: 92 - protein, 23 aa, synthetic construct
  REGION 1..23: note = Synthetic Peptide Mod-Kr1(58-81 Active Zone w Ile sub Cys and w Lys sub Cys)frag
  VARIANT 1: note = Xaa is Arg linked to NAc
  VARIANT 23: note = Xaa is Lys linked to N-epsilon-MPA
XPGNQKEAPWIFTLDENFKSDLX

SEQ ID NO: 93 - protein, 23 aa, synthetic construct
  REGION 1..23: note = Synthetic Peptide Mod-Kr1(58-81 Active Zone w Leu sub Cys and w Lys sub Cys)frag
  VARIANT 1: note = Xaa is Arg linked to NAc
  VARIANT 23: note = Xaa is Lys linked to N-epsilon-MPA
XPGNQKEAPWLFTLDENFKSDLX

SEQ ID NO: 94 - protein, 23 aa, synthetic construct
  REGION 1..23: note = Synthetic Peptide Mod-Kr1(58-80 Active Zone w Ala sub Cys)frag
  VARIANT 1: note = Xaa is Arg linked to NAc
XNPGNQKEAPWAFTLDENFKSDL

SEQ ID NO: 95 - protein, 23 aa, synthetic construct
  REGION 1..23: note = Synthetic Peptide Mod-Kr1(58-80 Active Zone w Val sub Cys)frag
  VARIANT 1: note = Xaa is Arg linked to NAc
XNPGNQKEAPWVFTLDENFKSDL

SEQ ID NO: 96 - protein, 23 aa, synthetic construct
  REGION 1..23: note = Synthetic Peptide Mod-Kr1(58-80 Active Zone w Ile sub Cys)frag
  VARIANT 1: note = Xaa is Arg linked to NAc
XNPGNQKEAPWIFTLDENFKSDL

SEQ ID NO: 97 - protein, 23 aa, synthetic construct
  REGION 1..23: note = Synthetic Peptide Mod-Kr1(58-80 Active Zone w Leu sub Cys)frag
  VARIANT 1: note = Xaa is Arg linked to NAc
XNPGNQKEAPWLFTLDENFKSDL

SEQ ID NO: 98 - protein, 23 aa, synthetic construct
  REGION 1..23: note = Synthetic Peptide Mod-Kr1(58-80 Active Zone w Ala sub Cys)frag
  VARIANT 1: note = Xaa is Arg linked to MPA
XNPGNQKEAPWAFTLDENFKSDL

SEQ ID NO: 99 - protein, 23 aa, synthetic construct
  REGION 1..23: note = Synthetic Peptide Mod-Kr1(58-80 Active Zone w Val sub Cys)frag
  VARIANT 1: note = Xaa is Arg linked to MPA
XNPGNQKEAPWVFTLDENFKSDL

SEQ ID NO: 100 - protein, 23 aa, synthetic construct
  REGION 1..23: note = Synthetic Peptide Mod-Kr1(58-80 Active Zone w Ile sub Cys)frag
  VARIANT 1: note = Xaa is Arg linked to MPA
XNPGNQKEAPWIFTLDENFKSDL

SEQ ID NO: 101 - protein, 23 aa, synthetic construct
  REGION 1..23: note = Synthetic Peptide Mod-Kr1(58-80 Active Zone w Leu sub Cys)frag
  VARIANT 1: note = Xaa is Arg linked to MPA
XNPGNQKEAPWLFTLDENFKSDL

SEQ ID NO: 102 - protein, 23 aa, synthetic construct
  REGION 1..23: note = Synthetic Peptide Mod-Kr1(58-80 Active Zone)
  VARIANT 1: note = Xaa is Arg linked to MPA-AEEA
XNPGNQKEAPWAFTLDENFKSDL

SEQ ID NO: 103 - protein, 23 aa, synthetic construct
  REGION 1..23: note = Synthetic Peptide Mod-Kr1(58-80 Active Zone)frag
  VARIANT 1: note = Xaa is Arg linked to MPA-AEEA
XNPGNQKEAPWVFTLDENFKSDL

SEQ ID NO: 104 - protein, 23 aa, synthetic construct
  REGION 1..23: note = Synthetic Peptide Mod-Kr1(58-80 Active Zone)frag
  VARIANT 1: note = Xaa is Arg linked to MPA-AEEA
XNPGNQKEAPWIFTLDENFKSDL

SEQ ID NO: 105 - protein, 23 aa, synthetic construct
  REGION 1..23: note = Synthetic Peptide Mod-Kr1(58-80 Active Zone)frag
  VARIANT 1: note = Xaa is Arg linked to MPA-AEEA
XNPGNQKEAPWLFTLDENFKSDL

SEQ ID NO: 106 - protein, 12 aa, synthetic construct
  REGION 1..12: note = Synthetic Peptide Mod-Kr1(58-69 Active Zone w Lys sub Cys)frag
  VARIANT 1: note = Xaa is Arg linked to NAc
XNPGNQKEAPWK

SEQ ID NO: 107 - protein, 12 aa, synthetic construct
  REGION 1..12: note = Synthetic Peptide Mod-Kr1(58-69 Active Zone w Lys sub Cys)frag
  VARIANT 1: note = Xaa is Arg linked to MPA
XNPGNQKEAPWK

SEQ ID NO: 108 - protein, 12 aa, synthetic construct
  REGION 1..12: note = Synthetic Peptide Mod-Kr1(58-69 Active Zone w Lys sub Cys)frag
  VARIANT 1: note = Xaa is Arg linked to MPA-AEEA
XNPGNQKEAPWK

SEQ ID NO: 109 - protein, 12 aa, synthetic construct
  REGION 1..12: note = Synthetic Peptide Mod-Kr1(58-69 Active Zone w Lys sub Cys)frag
  VARIANT 1: note = Xaa is Arg linked to NAc
  VARIANT 12: note = Xaa is Lys linked to N-epsilon-MPA
XNPGNQKEAPWX

SEQ ID NO: 110 - protein, 11 aa, synthetic construct
  REGION 1..11: note = Synthetic Peptide Mod-Kr1(58-68 Active Zone)frag
  VARIANT 1: note = Xaa is Arg linked to NAc
XNPGNQKEAPW

SEQ ID NO: 111 - protein, 11 aa, synthetic construct
  REGION 1..11: note = Synthetic Peptide Mod-Kr1(58-68 Active Zone)frag
  VARIANT 1: note = Xaa is Arg linked to MPA
XNPGNQKEAPW

SEQ ID NO: 112 - protein, 11 aa, synthetic construct
  REGION 1..11: note = Synthetic Peptide Mod-Kr1(58-68 Active Zone)frag
  VARIANT 1: note = Xaa is Arg linked to MPA-AEEA
XNPGNQKEAPW

SEQ ID NO: 113 - protein, 12 aa, synthetic construct
  REGION 1..12: note = Synthetic Peptide Mod-Kr1(70-81 Active Zone w Lys sub Cys)frag
  VARIANT 1: note = Xaa is Phe linked to NAc
XTLDENFKSDLK

SEQ ID NO: 114 - protein, 12 aa, synthetic construct
  REGION 1..12: note = Synthetic Peptide Mod-Kr1(70-81 Active Zone w Lys sub Cys)frag
  VARIANT 1: note = Xaa is Phe linked to MPA
XTLDENFKSDLK

SEQ ID NO: 115 - protein, 12 aa, synthetic construct
  REGION 1..12: note = Synthetic Peptide Mod-Kr1(70-81 Active Zone w Lys sub Cys)frag
  VARIANT 1: note = Xaa is Phe linked to MPA-AEEA
XTLDENFKSDLK

SEQ ID NO: 116 - protein, 12 aa, synthetic construct
  REGION 1..12: note = Synthetic Peptide Mod-Kr1(70-81 Active Zone w Lys sub Cys)frag
  VARIANT 1: note = Xaa is Phe linked to NAc
  VARIANT 12: note = Xaa is Lys linked to N-epsilon-MPA
XTLDENFKSDLX

SEQ ID NO: 117 - protein, 11 aa, synthetic construct
  REGION 1..11: note = Synthetic Peptide Mod-Kr1(70-80 Active Zone)frag
  VARIANT 1: note = Xaa is Phe linked to NAc
XTLDENFKSDL

SEQ ID NO: 118 - protein, 11 aa, synthetic construct
  REGION 1..11: note = Synthetic Peptide Mod-Kr1(70-80 Active Zone)frag
  VARIANT 1: note = Xaa is Phe linked to MPA
XTLDENFKSDL

SEQ ID NO: 119 - protein, 11 aa, synthetic construct
  REGION 1..11: note = Synthetic Peptide Mod-Kr1(70-80 Active Zone)frag
  VARIANT 1: note = Xaa is Phe linked to MPA-AEEA
XTLDENFKSDL

SEQ ID NO: 120 - protein, 10 aa, synthetic construct
  REGION 1..10: note = Synthetic Peptide Mod-Kr1(71-80 Active Zone)frag
  VARIANT 1: note = Xaa is Thr linked to NAc
XLDENFKSDL

SEQ ID NO: 121 - protein, 10 aa, synthetic construct
  REGION 1..10: note = Synthetic Peptide Mod-Kr1(71-80 Active Zone)frag
  VARIANT 1: note = Xaa is Thr linked to MPA
XLDENFKSDL

SEQ ID NO: 122 - protein, 10 aa, synthetic construct
  REGION 1..10: note = Synthetic Peptide Mod-Kr1(71-80 Active Zone)frag
  VARIANT 1: note = Xaa is Thr linked to MPA-AEEA
XLDENFKSDL

SEQ ID NO: 123 - protein, 9 aa, synthetic construct
  REGION 1..9: note = Synthetic Peptide Mod-Kr1(72-80 Active Zone)frag
  VARIANT 1: note = Xaa is Leu linked to NAc
XDENFKSDL

SEQ ID NO: 124 - protein, 9 aa, synthetic construct
  REGION 1..9: note = Synthetic Peptide Mod-Kr1(72-80 Active Zone)frag
  VARIANT 1: note = Xaa is Leu linked to MPA
XDENFKSDL

SEQ ID NO: 125 - protein, 9 aa, synthetic construct
  REGION 1..9: note = Synthetic Peptide Mod-Kr1(72-80 Active Zone)frag
  VARIANT 1: note = Xaa is Leu linked to MPA-AEEA
XDENFKSDL

SEQ ID NO: 126 - protein, 8 aa, synthetic construct
  REGION 1..8: note = Synthetic Peptide Mod-Kr1(73-80 Active Zone)frag
  VARIANT 1: note = Xaa is Asp linked to NAc
XENFKSDL

SEQ ID NO: 127 - protein, 8 aa, synthetic construct
  REGION 1..8: note = Synthetic Peptide Mod-Kr1(73-80 Active Zone)frag
  VARIANT 1: note = Xaa is Asp linked to MPA
XENFKSDL

SEQ ID NO: 128 - protein, 8 aa, synthetic construct
  REGION 1..8: note = Synthetic Peptide Mod-Kr1(73-80 Active Zone)frag
  VARIANT 1: note = Xaa is Asp linked to MPA-AEEA
XENFKSDL

SEQ ID NO: 129 - protein, 7 aa, synthetic construct
  REGION 1..7: note = Synthetic Peptide Mod-Kr1(73-79 Active Zone)frag
  VARIANT 1: note = Xaa is Asp linked to NAc
XENFKSD

SEQ ID NO: 130 - protein, 7 aa, synthetic construct
  REGION 1..7: note = Synthetic Peptide Mod-Kr1(73-79 Active Zone)frag
  VARIANT 1: note = Xaa is Asp linked to MPA
XENFKSD

SEQ ID NO: 131 - protein, 7 aa, synthetic construct
  REGION 1..7: note = Synthetic Peptide Mod-Kr1(73-79 Active Zone)frag
  VARIANT 1: note = Xaa is Asp linked to MPA-AEEA
XENFKSD

SEQ ID NO: 132 - protein, 85 aa, synthetic construct
  REGION 1..85: note = Synthetic Peptide Kr2(1-85 Active Zone)frag
  VARIANT 1: note = Xaa is Gly is linked to NAc
XRYHQCYNGSGMDYRGTASTTKSGHQCQPWALQHPHSHHLSSTDFPELGGGHAYCRNPGGQMEGPWCFTQNKNVRMELCDVPSCS

SEQ ID NO: 133 - protein, 85 aa, synthetic construct; identical to SEQ ID NO: 132
  REGION 1..85: note = Synthetic Peptide Kr2(1-85 Active Zone)frag
  VARIANT 1: note = Xaa is Gly is linked to MPA

SEQ ID NO: 134 - protein, 85 aa, synthetic construct; identical to SEQ ID NO: 132
  REGION 1..85: note = Synthetic Peptide Kr2(1-85 Active Zone)frag
  VARIANT 1: note = Xaa is Gly is linked to MPA-AEEA

SEQ ID NO: 135 - protein, 24 aa, synthetic construct
  REGION 1..24: note = Synthetic Peptide Mod-Kr2(56-79 Active Zone w Ala sub Cys and Lys sub Cys)frag
  VARIANT 1: note = Xaa is Arg linked to NAc
XNPGGQMEGPWAFTQNKNVRMELK

SEQ ID NO: 136 - protein, 24 aa, synthetic construct
  REGION 1..24: note = Synthetic Peptide Mod-Kr2(56-79 Active Zone w Val sub Cys and Lys sub Cys)frag
  VARIANT 1: note = Xaa is Arg linked to NAc
XNPGGQMEGPWVFTQNKNVRMELK

SEQ ID NO: 137 - protein, 24 aa, synthetic construct
  REGION 1..24: note = Synthetic Peptide Mod-Kr2(56-79 Active Zone w Ile sub Cys and Lys sub Cys)frag
  VARIANT 1: note = Xaa is Arg linked to NAc
XNPGGQMEGPWIFTQNKNVRMELK

SEQ ID NO: 138 - protein, 24 aa, synthetic construct
  REGION 1..24: note = Synthetic Peptide Mod-Kr2(56-79 Active Zone w Leu sub Cys and Lys sub Cys)frag
  VARIANT 1: note = Xaa is Arg linked to NAc
XNPGGQMEGPWLFTQNKNVRMELK

SEQ ID NO: 139 - protein, 24 aa, synthetic construct
  REGION 1..24: note = Synthetic Peptide Mod-Kr2(56-79 Active Zone w Ala sub Cys and Lys sub Cys)frag
  VARIANT 1: note = Xaa is Arg linked to MPA
XNPGGQMEGPWAFTQNKNVRMELK

SEQ ID NO: 140 - protein, 24 aa, synthetic construct
  REGION 1..24: note = Synthetic Peptide Mod-Kr2(56-79 Active Zone w Val sub Cys and Lys sub Cys)frag
  VARIANT 1: note = Xaa is Arg linked to MPA
XNPGGQMEGPWVFTQNKNVRMELK

SEQ ID NO: 141 - protein, 24 aa, synthetic construct
  REGION 1..24: note = Synthetic Peptide Mod-Kr2(56-79 Active Zone w Ile sub Cys and Lys sub Cys)frag
  VARIANT 1: note = Xaa is Arg linked to MPA
XNPGGQMEGPWIFTQNKNVRMELK

SEQ ID NO: 142 - protein, 24 aa, synthetic construct
  REGION 1..24: note = Synthetic Peptide Mod-Kr2(56-79 Active Zone w Leu sub Cys and Lys sub Cys)frag
  VARIANT 1: note = Xaa is Arg linked to MPA
XNPGGQMEGPWLFTQNKNVRMELK

SEQ ID NO: 143 - protein, 24 aa, synthetic construct
  REGION 1..24: note = Synthetic Peptide Mod-Kr2(56-79 Active Zone w Ala sub Cys and Lys sub Cys)frag
  VARIANT 1: note = Xaa is Arg linked to MPA-AEEA
XNPGGQMEGPWAFTQNKNVRMELK

SEQ ID NO: 144 - protein, 24 aa, synthetic construct
  REGION 1..24: note = Synthetic Peptide Mod-Kr2(56-79 Active Zone w Val sub Cys and Lys sub Cys)frag
  VARIANT 1: note = Xaa is Arg linked to MPA-AEEA
XNPGGQMEGPWVFTQNKNVRMELK

SEQ ID NO: 145 - protein, 24 aa, synthetic construct
  REGION 1..24: note = Synthetic Peptide Mod-Kr2(56-79 Active Zone w Ile sub Cys and Lys sub Cys)frag
  VARIANT 1: note = Xaa is Arg linked to MPA-AEEA
XNPGGQMEGPWIFTQNKNVRMELK

SEQ ID NO: 146 - protein, 24 aa, synthetic construct
  REGION 1..24: note = Synthetic Peptide Mod-Kr2(56-79 Active Zone w Leu sub Cys and Lys sub Cys)frag
  VARIANT 1: note = Xaa is Arg linked to MPA-AEEA
XNPGGQMEGPWLFTQNKNVRMELK

SEQ ID NO: 147 - protein, 24 aa, synthetic construct
  REGION 1..24: note = Synthetic Peptide Mod-Kr2(56-79 Active Zone w Ala sub Cys and Lys sub Cys)frag
  VARIANT 1: note = Xaa is Arg linked to NAc
  VARIANT 24: note = Xaa is Lys linked to N-epsilon-MPA
XNPGGQMEGPWAFTQNKNVRMELX

SEQ ID NO: 148 - protein, 24 aa, synthetic construct
  REGION 1..24: note = Synthetic Peptide Mod-Kr2(56-79 Active Zone w Val sub Cys and Lys sub Cys)frag
  VARIANT 1: note = Xaa is Arg linked to NAc
  VARIANT 24: note = Xaa is Lys linked to N-epsilon-MPA
XNPGGQMEGPWVFTQNKNVRMELX

SEQ ID NO: 149 - protein, 24 aa, synthetic construct
  REGION 1..24: note = Synthetic Peptide Mod-Kr2(56-79 Active Zone w Ile sub Cys and Lys sub Cys)frag
  VARIANT 1: note = Xaa is Arg linked to NAc
  VARIANT 24: note = Xaa is Lys linked to N-epsilon-MPA
XNPGGQMEGPWIFTQNKNVRMELX

SEQ ID NO: 150 - protein, 24 aa, synthetic construct
  REGION 1..24: note = Synthetic Peptide Mod-Kr2(56-79 Active Zone w Leu sub Cys and Lys sub Cys)frag
  VARIANT 1: note = Xaa is Arg linked to NAc
  VARIANT 24: note = Xaa is Lys linked to N-epsilon-MPA
XNPGGQMEGPWLFTQNKNVRMELX

SEQ ID NO: 151 - protein, 23 aa, synthetic construct
  REGION 1..23: note = Synthetic Peptide Mod-Kr2(56-78 Active Zone w Ala sub Cys)frag
  VARIANT 1: note = Xaa is Arg linked to NAc
XNPGGQMEGPWAFTQNKNVRMEL

SEQ ID NO: 152 - protein, 23 aa, synthetic construct
  REGION 1..23: note = Synthetic Peptide Mod-Kr2(56-78 Active Zone w Val sub Cys)frag
  VARIANT 1: note = Xaa is Arg linked to NAc
XNPGGQMEGPWVFTQNKNVRMEL

SEQ ID NO: 153 - protein, 23 aa, synthetic construct
  REGION 1..23: note = Synthetic Peptide Mod-Kr2(56-78 Active Zone w Ile sub Cys)frag
  VARIANT 1: note = Xaa is Arg linked to NAc
XNPGGQMEGPWIFTQNKNVRMEL

SEQ ID NO: 154 - protein, 23 aa, synthetic construct
  REGION 1..23: note = Synthetic Peptide Mod-Kr2(56-78 Active Zone w Leu sub Cys)frag
  VARIANT 1: note = Xaa is Arg linked to NAc
XNPGGQMEGPWLFTQNKNVRMEL

SEQ ID NO: 155 - protein, 23 aa, synthetic construct
  REGION 1..23: note = Synthetic Peptide Mod-Kr2(56-78 Active Zone w Ala sub Cys)frag
  VARIANT 1: note = Xaa is Arg linked to MPA
XNPGGQMEGPWAFTQNKNVRMEL

SEQ ID NO: 156 - protein, 23 aa, synthetic construct
  REGION 1..23: note = Synthetic Peptide Mod-Kr2(56-78 Active Zone w Val sub Cys)frag
  VARIANT 1: note = Xaa is Arg linked to MPA
XNPGGQMEGPWVFTQNKNVRMEL

SEQ ID NO: 157 - protein, 23 aa, synthetic construct
  REGION 1..23: note = Synthetic Peptide Mod-Kr2(56-78 Active Zone w Ile sub Cys)frag
  VARIANT 1: note = Xaa is Arg linked to MPA
XNPGGQMEGPWIFTQNKNVRMEL

SEQ ID NO: 158 - protein, 23 aa, synthetic construct
  REGION 1..23: note = Synthetic Peptide Mod-Kr2(56-78 Active Zone w Leu sub Cys)frag
  VARIANT 1: note = Xaa is Arg linked to MPA
XNPGGQMEGPWLFTQNKNVRMEL

SEQ ID NO: 159 - protein, 23 aa, synthetic construct
  REGION 1..23: note = Synthetic Peptide Mod-Kr2(56-78 Active Zone w Ala sub Cys)frag
  VARIANT 1: note = Xaa is Arg linked to MPA-AEEA
XNPGGQMEGPWAFTQNKNVRMEL

SEQ ID NO: 160 - protein, 23 aa, synthetic construct
  REGION 1..23: note = Synthetic Peptide Mod-Kr2(56-78 Active Zone w Val sub Cys)frag
  VARIANT 1: note = Xaa is Arg linked to MPA-AEEA
XNPGGQMEGPWVFTQNKNVRMEL

SEQ ID NO: 161 - protein, 23 aa, synthetic construct
  REGION 1..23: note = Synthetic Peptide Mod-Kr2(56-78 Active Zone w Ile sub Cys)frag
  VARIANT 1: note = Xaa is Arg linked to MPA-AEEA
XNPGGQMEGPWIFTQNKNVRMEL

SEQ ID NO: 162 - protein, 23 aa, synthetic construct
  REGION 1..23: note = Synthetic Peptide Mod-Kr2(56-78 Active Zone w Leu sub Cys)frag
  VARIANT 1: note = Xaa is Arg linked to MPA-AEEA
XNPGGQMEGPWLFTQNKNVRMEL

SEQ ID NO: 163 - protein, 12 aa, synthetic construct
  REGION 1..12: note = Synthetic Peptide Mod-Kr2(56-67 Active Zone w Lys sub Cys)frag
  VARIANT 1: note = Xaa is Arg linked to NAc
XNPGGQMEGPWK

SEQ ID NO: 164 - protein, 12 aa, synthetic construct
  REGION 1..12: note = Synthetic Peptide Mod-Kr2(56-67 Active Zone w Lys sub Cys)frag
  VARIANT 1: note = Xaa is Arg linked to MPA
XNPGGQMEGPWK

SEQ ID NO: 165 - protein, 12 aa, synthetic construct
  REGION 1..12: note = Synthetic Peptide Mod-Kr2(56-67 Active Zone w Lys sub Cys)frag
  VARIANT 1: note = Xaa is Arg linked to MPA-AEEA
XNPGGQMEGPWK

SEQ ID NO: 166 - protein, 12 aa, synthetic construct
  REGION 1..12: note = Synthetic Peptide Mod-Kr2(56-67 Active Zone w Lys sub Cys)frag
  VARIANT 1: note = Xaa is Arg linked to NAc
  VARIANT 12: note = Xaa is Lys linked to N-epsilon-MPA
XNPGGQMEGPWX

SEQ ID NO: 167 - protein, 11 aa, synthetic construct
  REGION 1..11: note = Synthetic Peptide Mod-Kr2(56-66 Active Zone)frag
  VARIANT 1: note = Xaa is Arg linked to NAc
XNPGGQMEGPW

SEQ ID NO: 168 - protein, 11 aa, synthetic construct
  REGION 1..11: note = Synthetic Peptide Mod-Kr2(56-66 Active Zone)frag
  VARIANT 1: note = Xaa is Arg linked to MPA
XNPGGQMEGPW

SEQ ID NO: 169 - protein, 11 aa, synthetic construct
  REGION 1..11: note = Synthetic Peptide Mod-Kr2(56-66 Active Zone)frag
  VARIANT 1: note = Xaa is Arg linked to MPA-AEEA
XNPGGQMEGPW

SEQ ID NO: 170 - protein, 12 aa, synthetic construct
  REGION 1..12: note = Synthetic Peptide Mod-Kr2(68-79 Active Zone w Lys sub Cys)frag
  VARIANT 1: note = Xaa is Phe linked to NAc
XTQNKNVRMELK

SEQ ID NO: 171 - protein, 12 aa, synthetic construct
  REGION 1..12: note = Synthetic Peptide Mod-Kr2(68-79 Active Zone w Lys sub Cys)frag
  VARIANT 1: note = Xaa is Phe linked to MPA
XTQNKNVRMELK

SEQ ID NO: 172 - protein, 12 aa, synthetic construct
  REGION 1..12: note = Synthetic Peptide Mod-Kr2(68-79 Active Zone w Lys sub Cys)frag
  VARIANT 1: note = Xaa is Phe linked to MPA-AEEA
XTQNKNVRMELK

SEQ ID NO: 173 - protein, 12 aa, synthetic construct
  REGION 1..12: note = Synthetic Peptide Mod-Kr2(68-79 Active Zone w Lys sub Cys)frag
  VARIANT 1: note = Phe is NAc-Phe
  VARIANT 12: note = Xaa is Lys linked to N-epsilon-MPA
XTQNKNVRMELX

SEQ ID NO: 174 - protein, 11 aa, synthetic construct
  REGION 1..11: note = Synthetic Peptide Mod-Kr2(68-78 Active Zone)frag
  VARIANT 1: note = Xaa is Phe linked to NAc
XTQNKNVRMEL

SEQ ID NO: 175 - protein, 11 aa, synthetic construct
  REGION 1..11: note = Synthetic Peptide Mod-Kr2(68-78 Active Zone)frag
  VARIANT 1: note = Xaa is Phe linked to MPA
XTQNKNVRMEL

SEQ ID NO: 176 - protein, 11 aa, synthetic construct
  REGION 1..11: note = Synthetic Peptide Mod-Kr2(68-78 Active Zone)frag
  VARIANT 1: note = Xaa is Phe linked to MPA-AEEA
XTQNKNVRMEL

SEQ ID NO: 177 - protein, 10 aa, synthetic construct
  REGION 1..10: note = Synthetic Peptide Mod-Kr2(69-78 Active Zone)frag
  VARIANT 1: note = Xaa is Thr linked to NAc
XQNKNVRMEL

SEQ ID NO: 178 - protein, 10 aa, synthetic construct
  REGION 1..10: note = Synthetic Peptide Mod-Kr2(69-78 Active Zone)frag
  VARIANT 1: note = Xaa is Thr linked to MPA
XQNKNVRMEL

SEQ ID NO: 179 - protein, 10 aa, synthetic construct
  REGION 1..10: note = Synthetic Peptide Mod-Kr2(69-78 Active Zone)frag
  VARIANT 1: note = Xaa is Thr linked to MPA-AEEA
XQNKNVRMEL

SEQ ID NO: 180 - protein, 9 aa, synthetic construct
  REGION 1..9: note = Synthetic Peptide Mod-Kr2(70-78 Active Zone)frag
  VARIANT 1: note = Xaa is Gln linked to NAc
XNKNVRMEL

SEQ ID NO: 181 - protein, 9 aa, synthetic construct
  REGION 1..9: note = Synthetic Peptide Mod-Kr2(70-78 Active Zone)frag
  VARIANT 1: note = Xaa is Gln linked to MPA
XNKNVRMEL

SEQ ID NO: 182 - protein, 9 aa, synthetic construct
  REGION 1..9: note = Synthetic Peptide Mod-Kr2(70-78 Active Zone)frag
  VARIANT 1: note = Xaa is Gln linked to MPA-AEEA
XNKNVRMEL

SEQ ID NO: 183 - protein, 8 aa, synthetic construct
  REGION 1..8: note = Synthetic Peptide Mod-Kr2(70-77 Active Zone)frag
  VARIANT 1: note = Xaa is Gln linked to NAc
XNKNVRME

SEQ ID NO: 184 - protein, 8 aa, synthetic construct
  REGION 1..8: note = Synthetic Peptide Mod-Kr2(70-77 Active Zone)frag
  VARIANT 1: note = Xaa is Gln linked to MPA
XNKNVRME

SEQ ID NO: 185 - protein, 8 aa, synthetic construct
  REGION 1..8: note = Synthetic Peptide Mod-Kr2(70-77 Active Zone)frag
  VARIANT 1: note = Xaa is Gln linked to MPA-AEEA
XNKNVRME

SEQ ID NO: 186 - protein, 93 aa, synthetic construct
  REGION 1..93: note = Synthetic Peptide Mod-Kr1(1-93 Active Zone)frag
  VARIANT 1: note = Xaa is Gly linked to NAc
XSNKNHKCYNSTGVDYRGTVSVTKSGRQCQPWNSQYPHTHTFTALRFPELNGGHSYCRNPGNQKEAPWCFTLDENFKSDLCDIPACDSKDSDI

SEQ ID NO: 187 - protein, 93 aa, synthetic construct; identical to SEQ ID NO: 186
  REGION 1..93: note = Synthetic Peptide Mod-Kr1(1-93 Active Zone)frag
  VARIANT 1: note = Xaa is Gly linked to MPA

SEQ ID NO: 188 - protein, 93 aa, synthetic construct; identical to SEQ ID NO: 186
  REGION 1..93: note = Synthetic Peptide Mod-Kr1(1-93 Active Zone)frag
  VARIANT 1: note = Xaa is Gly linked to MPA-AEEA

SEQ ID NO: 189 - protein, 91 aa, synthetic construct
  REGION 1..91: note = Synthetic Peptide K5(1-91 Active Zone)frag
GSSEEDCMFGNGKGYRGKRATTVTGTPCQDWAAQEPHRHSIFTPETNPRAGLEKNYCRNPDGDVGGPWCYTTNPRKLYDYCDVPQCAAPDI